(19) United States
(12) Patent Application Publication
(10) Pub. No.: US 2025/0160867 A1
(43) Pub. Date: May 22, 2025

(54) DEVICES AND RELATED METHODS TO TREAT CHRONIC OCCLUSIVE DVT

(71) Applicant: Koya Medical, Inc., Oakland, CA (US)

(72) Inventors: Anand Doraiswamy, Dallas, TX (US); Timothy W. Robinson, Sandown, NH (US); Amos G. Cruz, North Attleboro, MA (US); Anthony Maiorano, Franklin, MA (US); Jarren Baldwin, Oakland, CA (US)

(21) Appl. No.: 18/956,917

(22) Filed: Nov. 22, 2024

Related U.S. Application Data
(60) Provisional application No. 63/602,141, filed on Nov. 22, 2023.

(51) Int. Cl. A61B 17/221; A61B 17/00; A61B 17/22; A61B 17/32

(52) U.S. Cl. CPC A61B 17/221; A61B 17/00234; A61B 17/32; A61B 2017/00238; A61B 2017/00305; A61B 2017/22065; A61B 2017/2212

(57) ABSTRACT

This disclosure relates to thrombectomy devices and methods. The thrombectomy device can include a collection device, cutting device, and expansion device. The cutting device can be coupled to a proximal portion of the collection device. The expansion device can be coupled to a proximal portion of the cutting device. The expansion device can expand to expand the cutting device radially outward or collapse to collapse the cutting device radially inward. The expansion device can adjust and maintain a cutting diameter of the cutting device when cutting through a thrombus during proximal retraction of the thrombectomy device.

### CROSS REFERENCE

This application claims the priority benefit of U.S. Provisional Application No. 63/602,141, filed Nov. 22, 2023, which is hereby incorporated by reference in its entirety. All applications for which a foreign or domestic priority is identified in the Application Data Sheet as filed with the present application are hereby incorporated by reference under 37 CFR 1.57.

### FIELD

This disclosure relates to thrombectomy devices and methods to treat chronic occlusive deep vein thrombosis.

### BACKGROUND

The vascular system carries blood throughout the body. The vascular system includes arteries that distribute blood containing oxygen from the heart throughout the body and veins that carry deoxygenated blood back to the heart.

Thrombosis occurs when a thrombus (e.g., blood clot) forms within a blood vessel, whether venous or arterial. The thrombus may restrict blood flow through the blood vessel. For example, deep vein thrombosis (DVT) occurs when the thrombus forms in a deep vein, restricting blood flow back toward the heart. DVT typically develops in the lower leg, thigh, or pelvis but may also occur in other locations of the body, such as the arm. DVT can result in swelling, pain, discoloration, scaling, and/or ulcers. Additionally, a fragment (e.g., embolus) of the thrombus may break off and travel through the blood stream to the lungs, resulting in a pulmonary embolism (PE)—a potentially fatal condition.

### SUMMARY

Thrombi can be removed by way of a thrombectomy procedure. A thrombectomy procedure may include navigating a guide wire with the assistance of an imaging system (e.g., fluoroscopic x-ray imaging) through the vascular system to pass through the thrombus to a distal location. A catheter can be advanced along the guide wire and through the thrombus to deploy (e.g., expand) an expandable bag distal of the thrombus. The expandable bag can be retracted proximally, scraping the inner walls of the vessel, to capture the thrombus and deposit it into the catheter for removal.

A thrombus can include fibrin, red blood cells, platelets, leukocytes, and neutrophil extracellular traps. As time passes, the thrombus can become chronic, accumulating more collagen and fibrin content. As more collagen is accumulated, the chronic thrombus can become harder, which can make it more difficult to remove. For example, the chronic thrombus can be firmly attached to the vein wall. The chronic thrombus can be hard such that it can be difficult to core the chronic thrombus. For example, a proximally retracting bag can have difficulty passing through (e.g., cutting) the chronic thrombus to core (e.g., remove a middle portion) the thrombus.

Disclosed herein are thrombectomy devices that can, in some variants, address one or more of the problems discussed above. The thrombectomy devices can include a collection device, a cutting device, and and/or an expansion device. The cutting device can be coupled to a proximal portion of the collection device. A proximal portion of the cutting device can include one or more cutting features to cut a thrombus. The one or more cutting features can include an annular arrangement, which can provide a cutting diameter. The expansion device can be coupled to a proximal portion of the cutting device. The expansion device can collapse and expand the cutting device which, in some variants, can collapse and expand the collection device coupled to the cutting device. The expansion device can enable a user (e.g., clinician) to precisely adjust the cutting diameter provided by the one or more cutting features. The expansion device can maintain the one or more cutting features in a fixed arrangement during proximal retraction into a thrombus for cutting, which can help to prevent the cutting device from collapsing when cutting. For example, the expansion device can provide a high hoop stress and/or compression force across the cutting diameter to help prevent the cutting diameter provided by the cutting device from collapsing when encountering occlusive thrombus (e.g., chronic thrombus). In some variants, all or portions of the collection device, cutting device, and/or expansion device can be energized (e.g., heated and/or electrified) ease cutting through the thrombus, which can reduce the force required to cut through a thrombus and/or improve efficacy. In some variants, the thrombectomy device can include a conductor (e.g., wire) that can be heated, which can include heating with electrical energy (e.g., direct current, alternating current). In some variants, the conductor can be resistively heated with electricity. In some variants, the conductor can be coupled and/or otherwise disposed proximate any portion of the collection device, cutting device, and/or expansion device. The heated conductor can ease cutting a thrombus with heat. In some variants, the structure of the conductor itself can cut the thrombus. The conductor can form a loop which includes a portion carrying electrical energy distally and another portion carrying electrically energy proximally. In some variants, the one or more cutting features of the cutting device can be energized to ease cutting through the thrombus.

In use, a clinician can percutaneously access the vasculature of a patient. The clinician can navigate a guide wire through the vasculature to a thrombus, which can include using an imaging system (e.g., fluoroscopic x-ray imaging) to navigate. The guide wire can be distally advanced to penetrate and pass through the thrombus to position a distal end of the guide wire distal of the thrombus. In some variants, the guide wire can be energized with heat to ease penetration. A collapsed thrombectomy device, which can include being disposed within a tube such as a sheath, can be advanced distally over the guide wire to pass through and distal of the thrombus. With the collapsed thrombectomy device distal of the thrombus, the tube covering the collapsed thrombectomy device can be proximally retracted to uncover the collapsed thrombectomy device. The expansion device can be operated to expand the cutting device and/or the collection device. The expansion device can be operated to precisely adjust and maintain the cutting device at a desired cutting diameter (e.g., position the one or more cutting features of the cutting device in an arrangement for a desired cutting diameter), which can include expanding the cutting device to engage the vein wall or be offset radially inward of the vein wall. With the cutting device and/or collection device expanded, the thrombectomy device can be proximally retracted to engage the cutting features of the cutting device with the thrombus. The one or more cutting features can separate (e.g., cut) a portion (e.g., core) of the thrombus from the thrombus or substantially all of the thrombus from the vein wall. With the proximal retraction of the thrombectomy device, the separated portion of the thrombus or thrombus can pass through an interior of the cutting device and into an interior of the collection bag. With the cutting features of the cutting device proximal of the thrombus location, the expansion device can be operated to collapse the cutting device and collection device, which can trap the separated portion of thrombus or thrombus. The collapsed thrombus device can be proximally retracted out of the patient with the separated portion of the thrombus or thrombus. As described herein, in some variants, the thrombectomy device, can include a conductor that can be heated prior to and/or while retracting the one or more cutting features into the thrombus to ease cutting. Once through the thrombus, the conductor can stop being heated. In some variants, coring the thrombus can enable the vein to be more aptly treated with a stent.

In some aspects, the techniques described herein relate to a thrombectomy device configured to remove a thrombus from a vein, the thrombectomy device including: a plurality of shafts including an inner shaft, an inner expander shaft, and an outer expander shaft; a collection device including a basket with a closed distal end coupled to the inner shaft and an open proximal end; a cutting device including a frame, the frame including: an open distal end coupled to the open proximal end of the collection device; an open proximal end; an interior lumen spanning between the open proximal end and the open distal end of the frame; and one or more cutting elements disposed at the open proximal end; an expansion mechanism including a distal portion, a proximal portion, and a plurality of members spanning between the proximal portion and the distal portion, the distal portion coupled to the inner expander shaft, the proximal portion coupled to the outer expander shaft, and the plurality of members coupled to the one or more cutting elements; wherein the expansion mechanism is configured to expand the open proximal end of the cutting device with the proximal portion and distal portion moved toward each other such that the plurality of members move radially outward with the one or more cutting elements; and wherein the expansion mechanism is configured to collapse the open proximal end of the cutting device with the proximal portion and the distal portion moved away from each other such that the plurality of members move radially inward with the one or more cutting elements.

In some aspects, the techniques described herein relate to a thrombectomy device, wherein the inner expander shaft is configured to be distally advanced relative to the outer expander shaft to collapse the expansion device.

In some aspects, the techniques described herein relate to a thrombectomy device, wherein the inner expander shaft is configured to be proximally retracted relative to the outer expander shaft to expand the expansion device.

In some aspects, the techniques described herein relate to a thrombectomy device, wherein the one or more cutting elements include proximally-pointed cutting points.

In some aspects, the techniques described herein relate to a thrombectomy device, wherein the frame of the cutting device includes a plurality of struts coupled together to form diamond-shaped cells.

In some aspects, the techniques described herein relate to a thrombectomy device, wherein the collection device includes a distal tip that is tapered.

In some aspects, the techniques described herein relate to a thrombectomy device, wherein the one or more cutting elements includes four cutting elements.

In some aspects, the techniques described herein relate to a thrombectomy device, wherein the one or more cutting elements includes only four cutting elements.

In some aspects, the techniques described herein relate to a thrombectomy device, further including a conductor configured to be heated, the conductor disposed proximate the one or more cutting elements to ease cutting the thrombus.

In some aspects, the techniques described herein relate to a thrombectomy device, wherein the conductor is configured to be disposed at a periphery of the open proximal end of the frame of the cutting device.

In some aspects, the techniques described herein relate to a thrombectomy device, wherein the conductor is heated with electrical energy.

In some aspects, the techniques described herein relate to a thrombectomy device, wherein the outer expander shaft includes a handle and the inner expander shaft includes a handle.

In some aspects, the techniques described herein relate to a thrombectomy device, further including a threaded connection between the handle of the inner expander shaft and the handle of the outer expander shaft, wherein rotation of the handle of the inner expander shaft is configured to distally advance or proximally retract the inner expander shaft relative to the outer expander shaft.

In some aspects, the techniques described herein relate to a thrombectomy device including a collection device with a mesh structure having a closed distal end and an open proximal end; a cutting device including a frame, the frame including: an open distal end coupled to the open proximal end of the collection device; an open proximal end; an interior passage spanning between the open proximal end and the open distal end of the frame; and one or more cutting elements disposed at the open proximal end; an expander coupled to the open proximal end, the expander configured to expand and collapse to expand and collapse the open proximal end of the cutting device, wherein the expander is configured to enable a clinician to adjust and maintain a cutting diameter of the one or more cutting elements.

In some aspects, the techniques described herein relate to a thrombectomy device, wherein the expander is configured to enable the clinician to adjust the diameter of the cutting diameter along a continuum of diameters.

In some aspects, the techniques described herein relate to a thrombectomy device, wherein the expander includes a balloon that is configured to be inflated to expand the open proximal end of the cutting device and to be deflated to collapse the open proximal end of the cutting device.

In some aspects, the techniques described herein relate to a thrombectomy device, further including a conductor configured to be disposed proximate the one or more cutting elements, the conductor configured to be heated to facilitate cutting the thrombus.

In some aspects, the techniques described herein relate to a thrombectomy device, further including an inner expander shaft and an outer expander shaft, wherein the expander is coupled to the inner expander shaft and the outer expander shaft such that relative axial movement between the inner expander shaft and the outer expander shaft expand or collapse the expander.

In some aspects, the techniques described herein relate to a thrombectomy device, wherein the expander includes members configured to move radially outward with distal ends of the inner expander shaft and the outer expander shaft moved closer together.

In some aspects, the techniques described herein relate to a thrombectomy device, wherein the one or more cutting elements includes proximally directed cutting points.

In some aspects, the techniques described herein relate to a thrombectomy device, wherein the one or more cutting points provide the cutting diameter.

In some aspects, the techniques described herein relate to a method of removing a thrombus, the method including: unsheathing a thrombectomy device distal of a thrombus; proximally retracting an inner expander shaft relative to an outer expander shaft such that members of an expansion device coupled to the inner expander shaft and the outer expander shaft expand radially outward to expand a cutting diameter of a frame of a cutting device; proximally retracting the thrombectomy device to bring the cutting diameter of the frame in contact with the thrombus to cut the thrombus; receiving the cut thrombus through an interior lumen of the frame and into a collection basket; and distally advancing the inner expander shaft relative to the outer expander shaft such that members of the expansion device coupled to the inner expander shaft and the outer expander shaft collapse radially inward to collapse the cutting diameter of the frame.

In some aspects, the techniques described herein relate to a method, further including heating a conductor disposed proximate the cutting diameter.

In some aspects, the techniques described herein relate to a thrombectomy device including: a cutting device including a frame, the frame including: an open distal end; an open proximal end; an interior passage spanning between the open proximal end and the open distal end; and one or more cutting elements disposed at the open proximal end; an expander coupled to the open proximal end, the expander configured to expand and collapse to expand and collapse the open proximal end of the cutting device, wherein the expander is configured to enable a clinician to adjust and maintain a cutting diameter of the one or more cutting elements.

In some aspects, the techniques described herein relate to a thrombectomy device, wherein the expander includes a balloon that is configured to be inflated to expand the open proximal end of the cutting device and to be deflated to collapse the open proximal end of the cutting device.

In some aspects, the techniques described herein relate to a thrombectomy device, further including an inner expander shaft and an outer expander shaft, wherein the expander is coupled to the inner expander shaft and the outer expander shaft such that relative axial movement between the inner expander shaft and the outer expander shaft expands or collapses the expander.

In some aspects, the techniques described herein relate to a thrombectomy device, wherein the expander includes members configured to move radially outward with distal ends of the inner expander shaft and the outer expander shaft moved closer together.

In some aspects, the techniques described herein relate to a thrombectomy device, further including a conductor and a temperature sensor, the conductor disposed proximate the one or more cutting elements and configured to be heated, and the temperature sensor configured to detect temperature.

In some aspects, the techniques described herein relate to a thrombectomy device including: a collection device including mesh structure with a closed distal end and an open proximal end; and a cutting device including: a cylindrical structure including an open distal end, an open proximal end, and an inner passage extending between the open distal end and open proximal end of the cylindrical structure; a balloon disposed on the cylindrical structure, the balloon configured to expand and collapse; and a cutting element disposed at the proximal end of the cylindrical structure.

In some aspects, the techniques described herein relate to a thrombectomy device, wherein the cutting element includes an open truncated cone.

In some aspects, the techniques described herein relate to a thrombectomy device, wherein the cutting element includes a cutting ring.

In some aspects, the techniques described herein relate to a thrombectomy device including: a collection device including a mesh structure with a closed distal end and an open proximal end; and a cutting device including: an annular balloon including an open distal end and an open proximal end, the open distal end coupled to the open proximal end of the collection device; and a cutting ring disposed at the open proximal end of the annular balloon.

In some aspects, the techniques described herein relate to a thrombectomy device including: a collection device including a tapered mesh structure with a closed distal end and an open proximal end; a cutting device positioned proximally of the open proximal end of the collection device; and a sheath disposed between the collection device and the cutting device.

In some aspects, the techniques described herein relate to a thrombectomy device, further including a conductor configured to be disposed proximate a thrombus, wherein the conductor is configured to be heated.

In some aspects, the techniques described herein relate to a thrombectomy device including: one or more cutting elements; and an expander configured to expand and collapse to expand and collapse the one or more cutting elements, wherein the expander is configured to enable a clinician to adjust and maintain a cutting diameter of the one or more cutting elements.

In some aspects, the techniques described herein relate to a thrombectomy device, wherein the one or more cutting elements includes a plurality of proximally-directed prongs.

In some aspects, the techniques described herein relate to a thrombectomy device, further including an inner expander shaft and an outer expander shaft, wherein relative axial movement between the inner expander shaft and the outer expander shaft expands or collapses the expander.

In some aspects, the techniques described herein relate to a thrombectomy device, wherein distal advancement of the inner expander shaft relative to the outer expander shaft collapses the one or more cutting elements radially inward, and wherein proximal retraction of the inner expander shaft relative to the outer expander shaft expands the one or more cutting elements.

In some aspects, the techniques described herein relate to a thrombectomy device, wherein the expander includes a distal portion coupled to the inner expander shaft and a proximal portion coupled to the outer expander shaft.

In some aspects, the techniques described herein relate to a thrombectomy device, further including a plurality of members connected to and expanding between the distal portion and the proximal portion of the expander, wherein the plurality of members are configured to move radially outward when the distal portion and proximal portion of the expander are moved toward each other and move radially inward when then distal portion and proximal portion of the expander are moved away from each other.

In some aspects, the techniques described herein relate to a thrombectomy device, further including a conductor disposed proximate the one or more cutting elements, the conductor configured to be heated.

In some aspects, the techniques described herein relate to a thrombectomy device, wherein the conductor is configured to be heated with electrical energy.

In some aspects, the techniques described herein relate to a thrombectomy device, wherein the electrical energy is direct current.

In some aspects, the techniques described herein relate to a thrombectomy device, wherein the conductor is positioned along a cutting diameter of the one or more cutting elements.

In some aspects, the techniques described herein relate to a thrombectomy device, wherein the conductor is positioned along a periphery of an open proximal end of the one or more cutting elements.

In some aspects, the techniques described herein relate to a thrombectomy device, wherein the conductor is wire.

In some aspects, the techniques described herein relate to a thrombectomy device, further including a plurality of conductors, wherein the conductor is one of the plurality of conductors.

In some aspects, the techniques described herein relate to a thrombectomy device, further including a temperature sensor configured to sense temperature.

In some aspects, the techniques described herein relate to a thrombectomy device, wherein the conductor is configured to be resistively heated.

In some aspects, the techniques described herein relate to a thrombectomy device, further including a frame, wherein the one or more cutting elements are disposed at a proximal portion of the frame.

In some aspects, the techniques described herein relate to a thrombectomy device, wherein the frame includes a proximal open end, a distal open end, and an internal passage extending between the proximal open end and the distal open end.

In some aspects, the techniques described herein relate to a thrombectomy device, wherein the frame includes a plurality of struts forming diamond-shaped cells.

In some aspects, the techniques described herein relate to a thrombectomy device, further including a collection device coupled to the frame.

In some aspects, the techniques described herein relate to a thrombectomy device, further including a collection device.

In some aspects, the techniques described herein relate to a thrombectomy device, wherein the collection device is a mesh bag.

In some aspects, the techniques described herein relate to a thrombectomy device, wherein the collection device is a mesh basket.

In some aspects, the techniques described herein relate to a thrombectomy device including: a cutting device including a frame, the frame including: an open distal end; an open proximal end; and an interior passage spanning between the open proximal end and the open distal end; an expander coupled to the open proximal end, the expander configured to expand and collapse to expand and collapse the open proximal end of the cutting device, wherein the expander is configured to enable a clinician to adjust and maintain a diameter of the open proximal end.

In some aspects, the techniques described herein relate to a thrombectomy device, further including an inner expander shaft and an outer expander shaft, wherein relative axial movement between the inner expander shaft and the outer expander shaft expands or collapses the expander.

In some aspects, the techniques described herein relate to a thrombectomy device, wherein distal advancement of the inner expander shaft relative to the outer expander shaft collapses the open proximal end radially inward, and wherein proximal retraction of the inner expander shaft relative to the outer expander shaft expands the open proximal end.

In some aspects, the techniques described herein relate to a thrombectomy device, wherein the expander includes a distal portion coupled to the inner expander shaft and a proximal portion coupled to the outer expander shaft.

In some aspects, the techniques described herein relate to a thrombectomy device, further including a plurality of members connected to and expanding between the distal portion and the proximal portion of the expander, wherein the plurality of members are configured to move radially outward when the distal portion and proximal portion of the expander are moved toward each other and move radially inward when then distal portion and proximal portion of the expander are moved away from each other.

Neither the preceding summary nor the following detailed description purports to limit or define the scope of protection. The scope of protection is defined by the claims. Furthermore, reference is made herein to removing thrombi from veins. One of ordinary skill in the art will understand, after reviewing the entirety of this disclosure, that the systems and methods described herein may be applied to removing thrombi from arteries. Additionally, the systems and methods described herein can be used to remove other occlusions from the body.

### BRIEF DESCRIPTION OF THE DRAWINGS

The abovementioned and other features of the embodiments disclosed herein are described below with reference to the drawings of the embodiments. The illustrated embodiments are intended to illustrate, but not to limit, the scope of protection. Various features of the different disclosed embodiments can be combined to form further embodiments, which are part of this disclosure.

FIG. 1A illustrates a venous system in a limb.

FIG. 1B illustrates a vein with normal blood flow.

FIG. 1C illustrates a vein with a partial occlusion.

FIG. 1D illustrates a vein with a complete occlusion.

FIG. 2A illustrates a venous system in a limb.

FIG. 2B illustrates a vein with normal blood flow.

FIG. 2C illustrates a vein with early thrombus formation.

FIG. 2D illustrates a vein with a thrombus.

FIG. 2E illustrates an embolus breaking off of the thrombus.

FIG. 3A illustrates a venous system in a limb.

FIG. 3B illustrates a thrombus in a vein of the venous system causing swelling.

FIG. 4 illustrates a table showing the changes to a thrombus over time.

FIG. 5 illustrates a guidewire passing distally through a thrombus.

FIG. 6 illustrates a thrombectomy device including a collection device, cutting device, and expansion device.

FIG. 7A illustrates the expansion device expanding the cutting device of the thrombectomy system of FIG. 6.

FIG. 7B illustrates the expansion device collapsing the cutting device of the thrombectomy system of FIG. 6.

FIG. 8A illustrates an example expansion device of the thrombectomy system of FIG. 6.

FIG. 8B illustrates another example expansion device of the thrombectomy system of FIG. 6.

FIG. 9A illustrates a flat pattern for the expansion device illustrated in FIG. 8A.

FIG. 9B illustrates a flat pattern for an expansion device with three members for the thrombectomy system of FIG. 6.

FIG. 10 illustrates the cutting device of the thrombectomy system of FIG. 6.

FIG. 11 illustrates a flat pattern for the cutting device of the thrombectomy system of FIG. 10.

FIG. 12 illustrates the thrombectomy device of FIG. 6 sheathed in a collapsed configuration.

FIG. 13A illustrates an example proximal portion of the thrombectomy device of FIG. 6.

FIG. 13B illustrates an example proximal portion of the thrombectomy device of FIG. 6.

FIG. 14A illustrates a thermal device for the thrombectomy device of FIG. 6 or other thrombectomy devices described herein.

FIG. 14B illustrates an axial view of the thrombectomy device of FIG. 6 with a heated conductor.

FIG. 14C illustrates a perspective view of the thrombectomy device of FIG. 14B.

FIG. 14D illustrates an axial view of the thrombectomy device of FIG. 6 with a plurality of heated conductors.

FIG. 14E illustrates a perspective view of the thrombectomy device of FIG. 14D.

FIG. 15A illustrates an example expansion device for the thrombectomy system of FIG. 6 in a collapsed configuration.

FIG. 15B illustrates the example expansion device of FIG. 15A in an expanded configuration.

FIG. 16A illustrates an example expansion device for the thrombectomy system of FIG. 6 in a collapsed configuration.

FIG. 16B illustrates the example expansion device of FIG. 16A in an expanded configuration.

FIG. 17A illustrates an example expansion device for the thrombectomy system of FIG. 6 in a collapsed configuration.

FIG. 17B illustrates the example expansion device of FIG. 17A in an expanded configuration.

FIG. 18 illustrates an example expansion device for the thrombectomy system of FIG. 6.

FIG. 19A illustrates an example expansion device with a balloon for the thrombectomy system of FIG. 6 with an inner shaft off a central axis of the cutting device.

FIG. 19B illustrates the example expansion device of FIG. 19A with an example cutting device with features to center the inner shaft.

FIG. 19C illustrates an axial view of the example expansion device and cutting device of FIG. 19B.

FIG. 20A illustrates a thrombectomy device with a collection device and coring device in a collapsed configuration.

FIG. 20B illustrates the thrombectomy device of FIG. 20A with the collection device expanded and a balloon of the cutting device expanded.

FIG. 21A illustrates a thrombectomy device with a collection device and a cutting device with a cutting element having a conical frustum shape.

FIG. 21B illustrates a thrombectomy device with a collection device and a cutting element with a cutting ring.

FIG. 22A illustrates a thrombectomy device with a collection device and a cutting device with a balloon and angled cutting ring.

FIG. 22B illustrates a cutting device that has a balloon with an inflation channel arranged in a serpentine configuration.

FIG. 22C illustrates a cutting device that has a balloon with an inflation channel arranged in a coiled configuration.

FIG. 23 illustrates a thrombectomy device with a collection device, a cutting device with a mesh cutting basket, and a sleeve.

FIG. 24A illustrates a side view of a thrombectomy device with a collection device, cutting device with blades, and a sleeve.

FIG. 24B illustrates a perspective view of the thrombectomy device of FIG. 25A.

FIG. 25 illustrates a thrombectomy device with a collection device and cutting device with an inner mesh.

FIG. 26A illustrates a perspective view of a cutting device.

FIG. 26B illustrates a side view of the cutting device of FIG. 26A.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below. Furthermore, this disclosure describes many embodiments in reference to veins and arteries; the systems and methods described in relation to veins can be applied to arteries and those described in relation to arteries can be applied to veins.

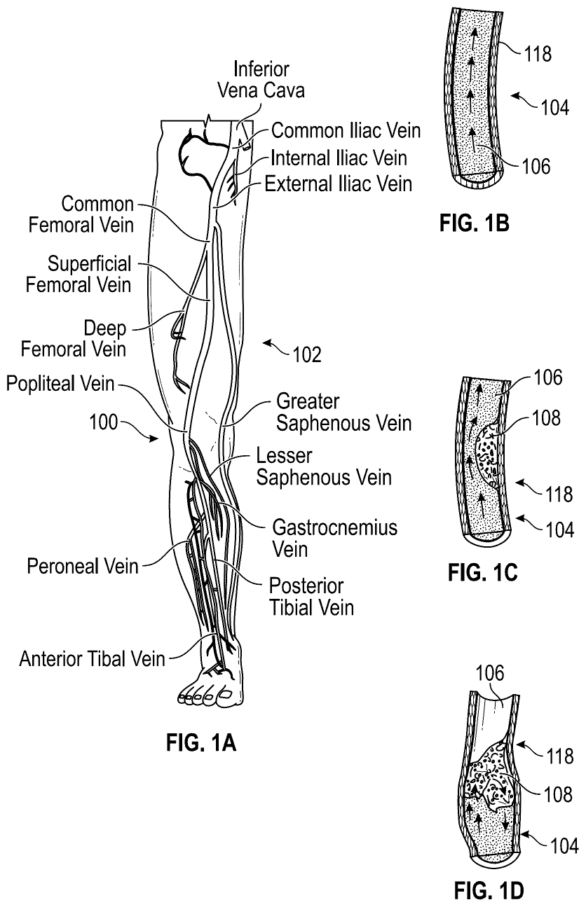

FIG. 1A illustrates a limb 100 (e.g., leg, arm) of a person. The limb 100 includes a venous system 102 that carries deoxygenated blood from the limb 100 back to the heart.

FIG. 1B illustrates a cross-section of a portion of a vein 104 of the venous system 102 with normal blood flow. The vein 104 includes a flow path 106 for the deoxygenated blood bounded by the wall 118 of the vein 104. A thrombus 108 may develop within a vein 104, as illustrated in FIG. 1C, to partially restrict (e.g., impede, occlude, etc.) blood flow through the 106. The thrombus 108 may form on the wall 118 of the vein 104, which may include a valve. The wall 118 may from along the wall 118 for a variety of reasons, which may include damage to the wall 118, inactivity, diet, and/or other reasons. The thrombus 108 may grow in size to completely block (e.g., impede, occlude, etc.) blood flow through the flow path 106, as shown in FIG. 1D. The occlusion of the blood flow can result in swelling, pain, and/or discoloration of the limb 100. The removal of thrombi can relieve a patient of the foregoing symptoms as well as prevent or at least slow further symptoms from developing.

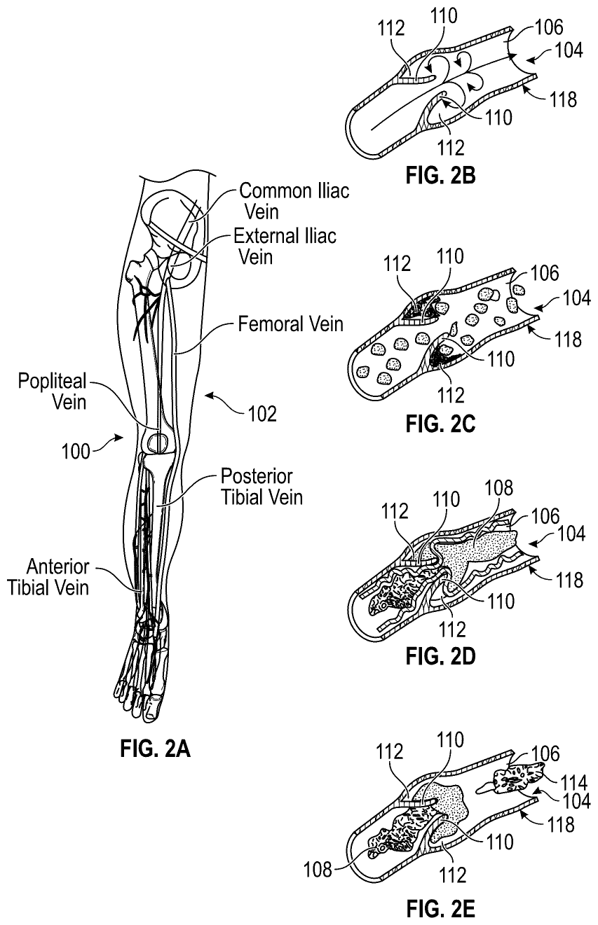

FIG. 2A illustrates a limb 100 (e.g., leg, arm) of a person. The limb 100 includes venous system 102 that carries deoxygenated blood from the limb 100 back to the heart.

FIG. 2B illustrates a cross-section of a portion of a vein 104 of the venous system 102 with normal blood flow. The vein 104 includes a flow path 106 for the deoxygenated blood bounded by the wall 118 of the vein 104. The vein 104 may include valves 110. The valves 110 may open to allow blood to flow toward the heart and close to prevent backflow. Pockets 112 can be disposed downstream of the valves 110 between the valves 110 and the wall 118 of the vein 104. As shown, a portion of blood in the flow path 106 may flow into the pockets 112. A thrombus may begin to develop on the wall 118 of the vein 104, valves 110, and/or in the pockets 112, as shown in FIG. 2C, until a thrombus 108 is formed, as illustrated in FIG. 2D. The thrombus 108 may fill the flow path 106, which can include extending around the valves 110 and into the pockets 112. The thrombus 108 may prevent proper function of the valves 110. The thrombus 108 may partially or completely block blood flow through the vein 104. In some instances, an embolus 114 may break off from the thrombus 108, as illustrated in FIG. 2E. The embolus 114 may travel to other regions of the body, which can include traveling to the lungs and result in a pulmonary embolism (PE) a potentially lethal condition. Accordingly, the removal of thrombi may help a patient avoid a lethal condition.

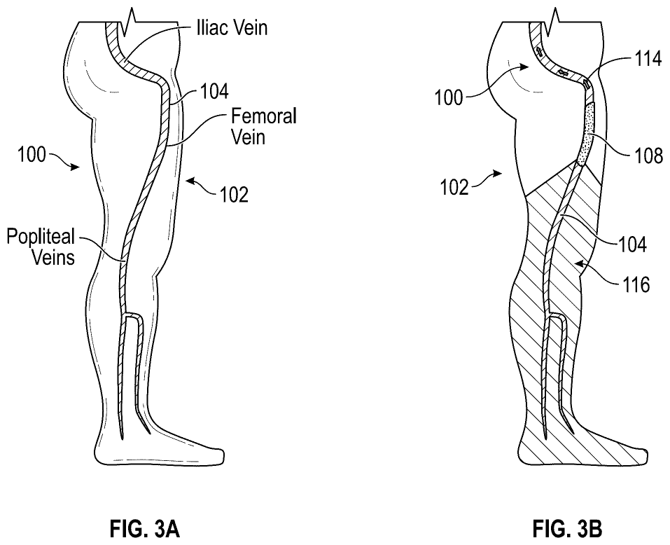

FIG. 3A illustrates a limb 100 (e.g., leg, arm) of a person. The limb 100 includes a venous system 102 with multiple veins 104. As shown in FIG. 3B, a thrombus 108 may form in the vein 104 to block blood flow. The blocked blood flow may prevent drainage of blood from the limb 100 and cause swelling of the limb 100 below the thrombus 108 as indicated by portion 116. As detailed herein, an embolus 114 may separate from the thrombus 108 and travel to other regions of the body, which can include traveling to the lungs and result in a pulmonary embolism (PE).

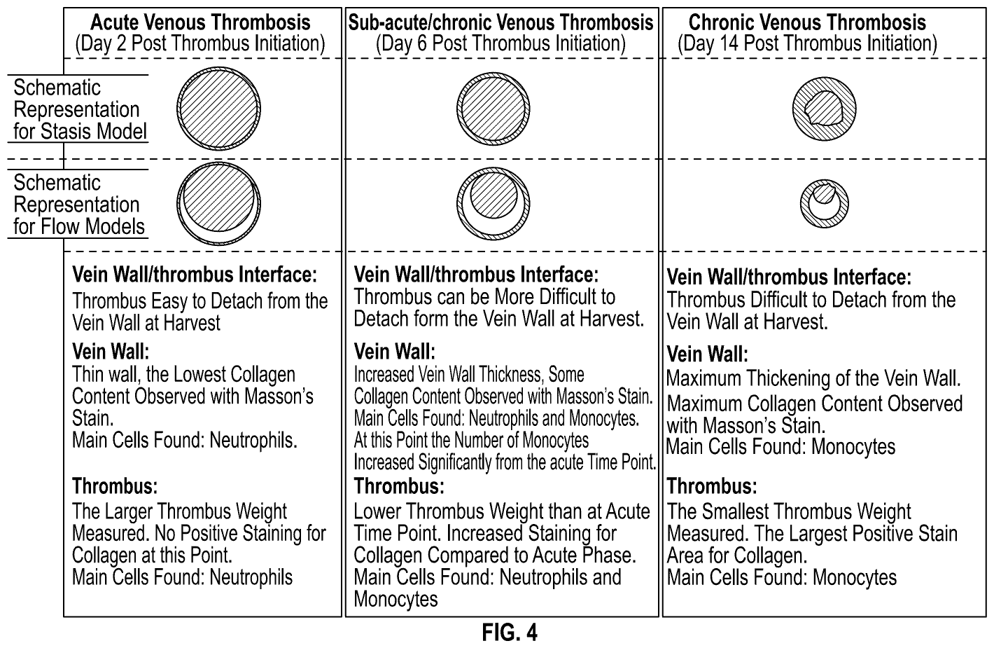

As a thrombus ages, the characteristics of the proximate portion of the vein and the thrombus itself may change, as indicated in the table shown in FIG. 4.

For example, initially after formation (e.g., two days post thrombus initiation), a thrombus may be referred to as an acute thrombus. The vein wall proximate the acute thrombus may be thin and have a low collagen content. The main cells found in the vein wall proximate the acute thrombus may be neutrophils. The acute thrombus itself may have no or relatively little collagen. The main cells found in the acute thrombus may be neutrophils. The acute thrombus may be readily detached from the vein wall and/or penetrated.

After some additional time (e.g., six days post thrombus initiation), a thrombus may be referred to as a sub-acute/chronic thrombus. The vein wall proximate the sub-acute/chronic thrombus may be thickened and have a higher collagen content compared to the acute thrombus period. The main cells found in the vein wall proximate the sub-acute/chronic thrombus may be neutrophils and monocytes with the quantity of monocytes significantly increased compared to the acute thrombus period. The sub-acute/chronic thrombus itself may have an increased collagen content compared to the acute thrombus. The main cells found in the sub-acute/chronic thrombus may be neutrophils and monocytes. The sub-acute/chronic thrombus may be more difficult to detach from the vein wall compared to the acute thrombus and/or more difficult to penetrate. The sub-acute/chronic thrombus may have a lower weight compared to the acute thrombus.

After some additional time (e.g., fourteen days post thrombus initiation), a thrombus may be referred to as a chronic thrombus. The vein wall proximate the chronic thrombus may be thickened and have a higher collagen content compared to the sub-acute/chronic thrombus period. The main cells found in the vein wall proximate the chronic thrombus may be monocytes. The chronic thrombus may have an increased collagen content compared to the sub-acute/chronic thrombus. The main cells found in the chronic thrombus may be monocytes. The chronic thrombus may be more difficult to detach from the vein wall compared to the sub-acute/chronic thrombus and/or more difficult to penetrate. The chronic thrombus may have a lower weight compared to the sub-acute/chronic thrombus.

The hardness of a thrombus may increase over time, which may be due to the increase in collagen content. For example, a chronic thrombus may be harder than a sub-acute/chronic thrombus which may be harder than an acute thrombus. A harder thrombus may be more difficult to penetrate, which can make removal more difficult. Additionally, as indicated above, a chronic thrombus may be harder to detach from the vein wall compared to the sub-acute/chronic thrombus which may be harder to detach from the vein wall compared to the acute thrombus.

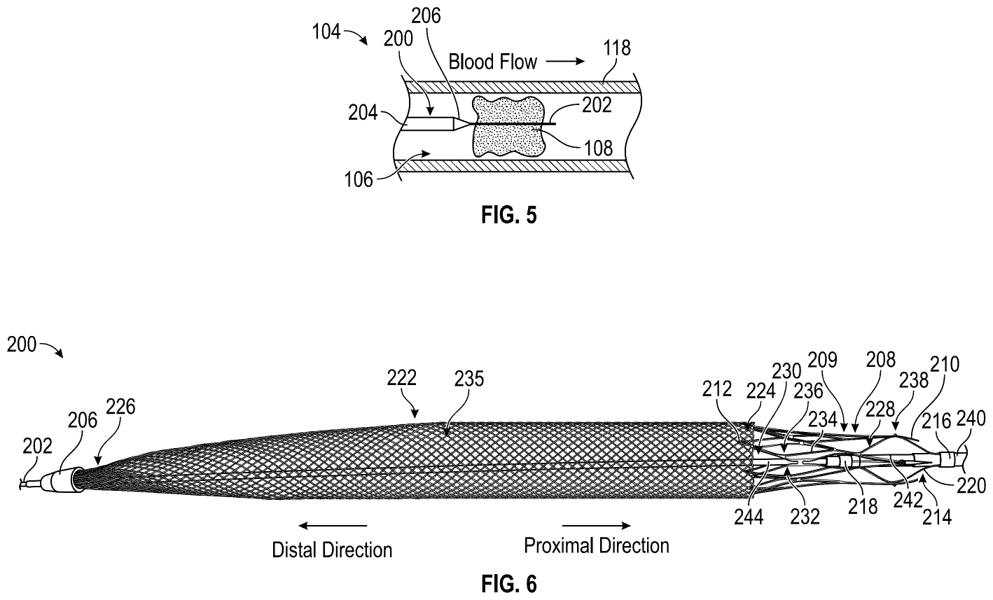

To remove a thrombus, the vasculature of a patient can be accessed percutaneously. A guide wire 202 can be navigated through the vasculature to a thrombus 108 disposed inside a vein 104. The thrombus 108 can be attached to a wall 118 of the vein 104. As illustrated in FIG. 5, guide wire 202 can penetrate and pass through the thrombus 108 to position a distal end of the guide wire 202 distal of the thrombus 108. In some variants, the guide wire 202 can include energized features, such as heated and/or electrified features, to ease penetrating and passing through the thrombus 108. With the guide wire 202 positioned, a thrombectomy device 200 can be advanced distally over the guide wire 202 to the location of the thrombus 108.

The thrombectomy device 200 can include a tip 206 (e.g., distal tip), which can be tapered and/or bullet shaped. The thrombectomy device 200 can include a sheath 204, which can also be referred to as an outer tube. Components of the thrombectomy device 200 (e.g., collapsed components of the thrombectomy device 200) can be covered by the sheath 204 to ease delivery. The tip 206 can be disposed at the distal end of the sheath 204, which can close the distal end of the sheath 204. The thrombectomy device 200 can be advanced distally over the guide wire 202 through the thrombus 108 to a position distal of the thrombus 108. With the thrombectomy device 200 distal of the thrombus 108, the sheath 204 can be retracted to uncover portions of the thrombectomy device 200, which can include collapsed components of the thrombectomy device 200.

FIG. 6 illustrates a thrombectomy device 200, which can also be referred to as a thrombectomy system. The thrombectomy device 200 can include a collection device 222, a cutting device 208, and/or expansion device 214. The cutting device 208 can be used to cut a thrombus 108 from a wall 118 of a vein 104 and/or separate a portion (e.g., central core) of a thrombus 108 for removal with proximal retraction of the cutting device 208 into the thrombus 108. The expansion device 214 can be coupled to the cutting device 208 to set and/or maintain a cutting diameter of the cutting device 208. The expansion device 214 can be expanded or collapsed to expand or collapse the cutting device 208. The collection device 222 can be coupled to the cutting device 208 to catch the thrombus or separated portion of the thrombus 108 cut by the cutting device 208. The expansion of the cutting device 208 can cause the expansion of the collection device 222. The expansion device 214 can convert axial movement into radial expansion, which can radially expand the cutting device 208 and/or collection device 222.

The collection device 222 can also be referred to as a bag, basket, mesh bag, mesh basket, and/or mesh structure. The collection device 222 can include a closed distal end 226 and/or an open proximal end 224. The collection device 222 can include a tapered shape, which can include tapering from the open proximal end 224 to the closed distal end 226. The collection device 222 can include a tubular structure with an open proximal end 224 and closed distal end 226. The collection device 222 can include a mesh structure made from woven filaments. The collection device 222 can include an interior 235 that is accessible from the open proximal end 224. The open proximal end 224 can include a circular or oval opening. The collection device 222 can include the tip 206. The tip 206, which can include a tapered and/or bullet shape, can be disposed at the closed distal end 226. The closed distal end 226 and/or tip 206 can be coupled to an inner shaft 244 (e.g., distal portion of inner shaft 244), which can also be referred to as an inner tube, inner catheter, collection device tube, and/or collection device shaft.

The cutting device 208 can also be referred to as a coring device, cutter, corer, and/or cylindrical structure. The cutting device 208 can include a frame 209. The frame 209 can have a cylindrical and/or tubular shape. The frame 209 can include a plurality of struts 234, which can also be referred to as members. The plurality of struts 234 can be arranged to form a plurality of cells 236. The plurality of cells 236 can include diamond shapes. The cutting device 208 can include an open distal end 230, open proximal end 228, and/or interior lumen 232 extending from the open proximal end 228 to the open distal end 230. The open distal end 230 can be coupled to the open proximal end 224 of the collection device 222. In some variants, the open distal end 230 of the cutting device 208 can couple to the collection device 222 at a position distal of the open proximal end 224 of the collection device 222. The open distal end 230 of the frame 209 can include distal attachment portions 212 that can be coupled to the collection device 222, which can include the open proximal end 224 of the collection device 222. In some variants, the frame 209 can be fastened (e.g., tied), welded, and/or adhered to the collection device 222 (e.g., open proximal end 224 of the collection device 222). In some variants, the frame 209 can be tied and then welded to the collection device 222 (e.g., open proximal end 224 of the collection device 222). In some variants, the cutting device 208 and collection device 222 can be integrally formed. The cutting device 208 can include one or more cutting elements 210, which can include cutting points, sharp points, cutting edges, spikes, prongs, and/or blades. The one or more cutting elements 210 can be disposed at the open proximal end 228 of the frame 209. The one or more cutting elements 210 can be arranged in an annular shape, which can include being distributed circumferentially along the annular shape. The one or more cutting elements 210 can provide a cutting diameter. The one or more cutting elements 210 can be oriented (e.g., point) in a proximal direction to cut a thrombus 108 with proximal retraction of the cutting device 208 into the thrombus 108. The cutting device 208 can include one, two, three, four, five, six, seven, eight, nine, ten, eleven, twelve, thirteen, fourteen, fifteen, sixteen, seventeen, eighteen, nineteen, or twenty or more cutting elements 210.

The expansion device 214 can also be referred to as an expander, expansion mechanism, and/or malecot. The expansion device 214 can convert axial movement into radial expansion. The expansion device 214 can include a distal portion 218, which can be an annular structure, ring, and/or band. The distal portion 218 can be coupled to (e.g., disposed on) an inner expander shaft 242, which can also be referred to as an inner expander tube and/or inner expander catheter. The inner shaft 244 can be disposed through the inner expander shaft 242. The inner expander shaft 242 and inner shaft 244 can be moveable relative to each other. The expansion device 214 can include a proximal portion 216, which can be an annular structure, ring, and/or band. The proximal portion 216 can be coupled to (e.g., disposed on) an outer expander shaft 240, which can also be referred to as an outer expander tube and/or outer expander catheter. The inner expander shaft 242 can be disposed through the outer expander shaft 240. The outer expander shaft 240 and inner expander shaft 242 can be moveable relative to each other. The expansion device 214 can include a plurality of members 220, which can also be referred to as struts, cross-pieces, supports, bands, ribbons, and/or wings. The plurality of members 220 can connect to and extend between the proximal portion 216 and the distal portion 218. The plurality of members 220 can include one, two, three, four, five, six, seven, eight, nine, ten, eleven, twelve, thirteen, fourteen, fifteen, sixteen, seventeen, eighteen, nineteen, or twenty or more members 220. The number of members 220 can correspond to the number of cutting elements 210. The plurality of members 220 can curve radially outward from the proximal portion 216 to a vertex 238 and then curve radially inward to the distal portion 218. The cutting elements 210, which can include a portion of the frame 209 proximate the cutting elements 210, can be coupled (e.g., fastened, tied, welded, adhered) to the plurality of members 220. The cutting elements 210 can be coupled to the plurality of members 220 at the vertices 238 of the members 220. In some variants, the cutting elements 210 can be angled inward (e.g. acute angle) to avoid cutting the wall 118 of the vein 104. The cutting elements 210 can be angled inward away from the wall 118. The angle of the cutting elements 210 can also help with reentry into the sheath 204. In some variants, the cutting elements 210 can be coupled to the plurality of members 220 proximally of the vertices 238, which can angle the cutting elements 210 inward.

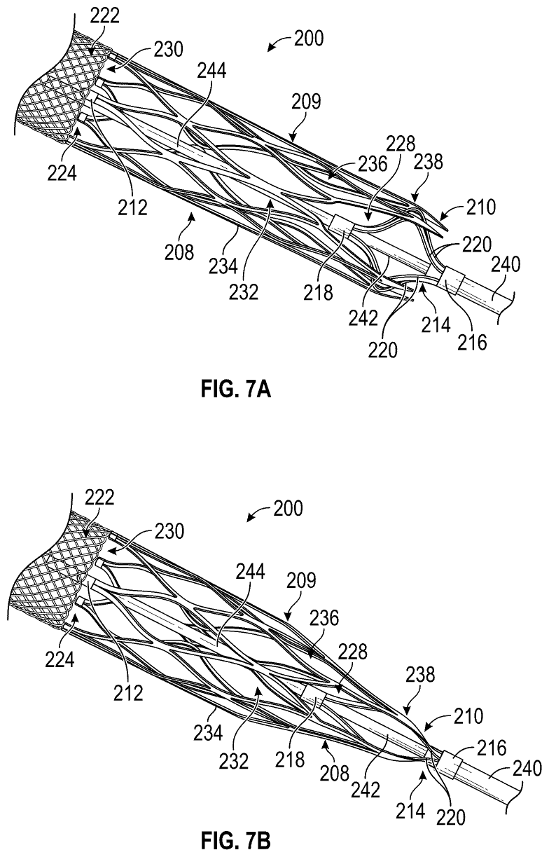

As illustrated in FIG. 7A, the expansion device 214 can expand, which can expand the cutting device 208 to enlarge the cutting diameter provided by the cutting elements 210. The expansion of the cutting device 208 can expand the collection device 222. To expand the expansion device 214, the proximal portion 216 and distal portion 218 can be moved toward each other, causing the plurality of members 220 to move radially outward with the cutting elements 210 coupled to the plurality of members 220. For example, the inner expander shaft 242 with the distal portion 218 coupled thereto can be proximally retracted relative to the outer expander shaft 240 to move the distal portion 218 toward the proximal portion 216 coupled to the outer expander shaft 240. In some variants, the outer expander shaft 240 with the proximal portion 216 coupled thereto can be distally advanced relative to the inner expander shaft 242 to move the proximal portion 216 toward the distal portion 218 coupled to the inner expander shaft 242.

AS illustrated in FIG. 7B, the expansion device 214 can collapse, which can collapse the cutting device cutting device 208 to reduce the cutting diameter or even close the cutting diameter provided by the cutting elements 210. The collapse of the cutting device 208 can collapse the collection device 222. To collapse the expansion device 214, the proximal portion 216 and distal portion 218 can be moved away from each other, causing the plurality of members 220 to move radially inward with the cutting elements 210 coupled to the plurality of members 220. For example, the inner expander shaft 242 with the distal portion 218 coupled thereto can be distally advanced relative to the outer expander shaft 240 to move the distal portion 218 away from the proximal portion 216 coupled to the outer expander shaft 240. In some variants, the outer expander shaft 240 with the proximal portion 216 coupled thereto can be proximally retracted relative to the inner expander shaft 242 to move the proximal portion 216 away from the distal portion 218 coupled to the inner expander shaft 242.

A clinician can adjust the cutting diameter provided by the cutting elements 210 along a continuum of diameters by adjusting the distance between the proximal portion 216 and distal portion 218. In some variants, the outer expander shaft 240 and/or inner expander shaft 242 can include markings to indicate relative positioning between the outer expander shaft 240 and inner expander shaft 242 that corresponds to certain cutting diameters. In some variants, the thrombectomy device 200 can include one or more features to facilitate stepped (e.g., incremental) adjustment of the diameters.

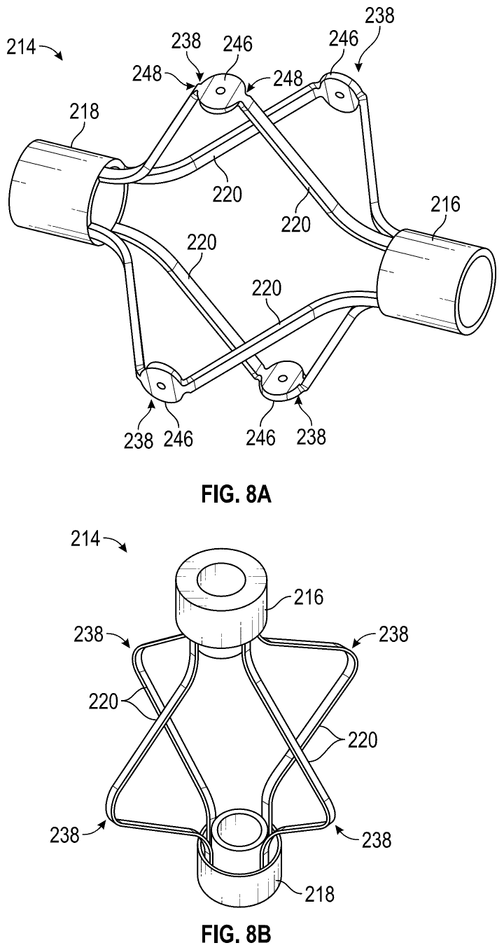

FIG. 8A illustrates an example expansion device 214. As illustrated, the expansion device 214 can include the proximal portion 216, which can be an annular structure sized to be disposed on (e.g., coupled to) the outer expander shaft 240. The expansion device 214 can include the distal portion 218, which can be an annular structure sized to be disposed on (e.g., coupled to) the inner expander shaft 242. The expansion device 214 can include a plurality of members 220 connecting and extending between the proximal portion 216 and distal portion 218. The plurality of members 220 can be circumferentially distributed about the axis of the expansion device 214, which can include being circumferentially distributed evenly. The plurality of members 220 can include cross-sections of varying shapes (e.g., polygon such as square and/or rectangle) and/or sizes. The plurality of members 220 can curve radially outward from the proximal portion 216 to vertices 238 and radially inward from the vertices 238 to the distal portion 218. The cutting elements 210 and/or features of the frame 209 proximate the cutting elements 210 can be coupled to the vertices 238 and/or proximal of the vertices 238 to help angle the cutting elements 210 inward. The plurality of members 220 can include features to facilitate coupling to the cutting elements 210 and/or features of the frame 209 proximate the cutting elements 210. For example, the plurality of members 220 can include attachment portions 246, which can also be referred to as anchor points. The attachment portions 246 can include an enlarged size relative to the other portions of the plurality of members 220. The attachment portions 246 can include flanges to provide a larger surface area for coupling. The attachment portions 246 can include one or more apertures to facilitate coupling. The plurality of members 220 can include flex portions 248, which can include a reduced size relative to the other portions of the plurality of members 220, adjacent the attachment portions 246. The flex portions 248 can facilitate orienting the attachment portions 246. In some variants, the plurality of members 220 can omit the flex portions 248 and/or vertices 238 as illustrated in FIG. 8B. In some variants, the plurality of members 220 can include a consistent cross-section. In some variants, the distal portion 218 and/or proximal portion 216 can include annular gaps to receive ends of the plurality of members 220.

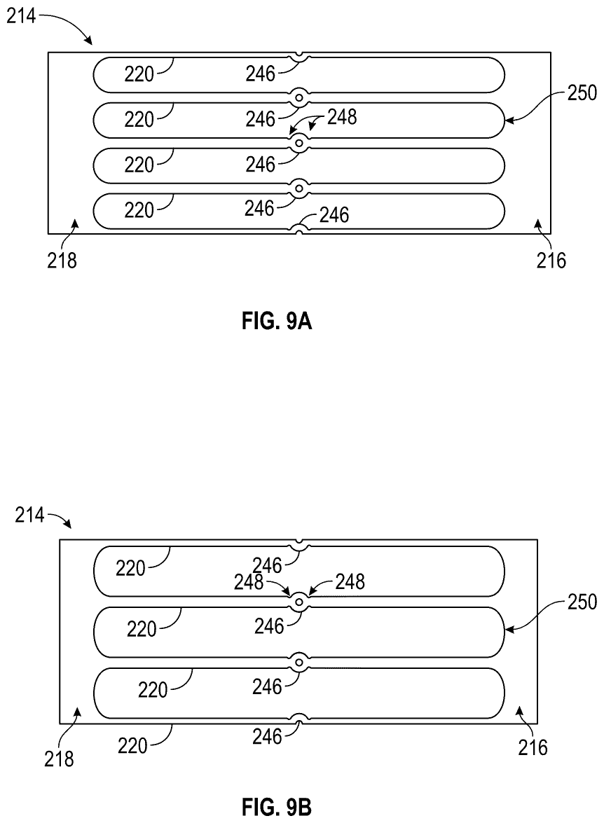

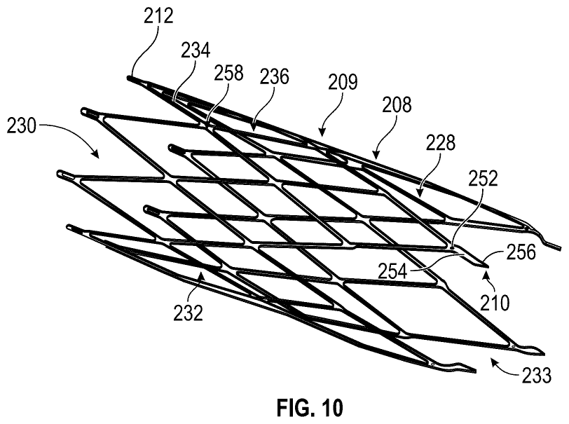

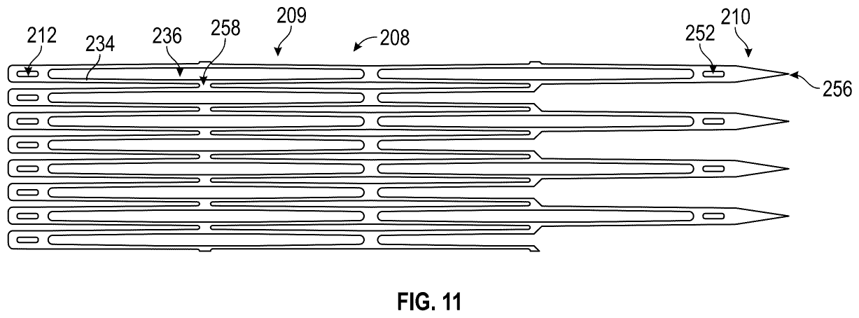

FIG. 9A illustrates a flat pattern for an expansion device 214 similar to that illustrated in FIG. 8A. The flat pattern can be cut from a sheet of material, which can include Nitinol. Curved portions 250 can be disposed in the proximal portion 216 and/or distal portion 218 spanning between adjacent members 220. In some variants, one of the plurality of members 220 can be divided in half in an axial direction as illustrated.

To form the assembled expansion device 214, the flat pattern for the expansion device 214 can be formed (e.g., rolled) into a tubular structure, which can include being formed (e.g., rolled) around a cylindrical structure. The halved member 220 can be coupled together, which can at least include welding and/or adhering, to hold the expansion device 214 in the tubular shape. A fixture, such as a disk fixture can be disposed between the proximal portion 216 and distal portion 218 to push the plurality of members 220 radially outward. The fixture can be positioned at the attachment portions 246 to form the vertices 238 at the attachment portions 246. In some variants, the expansion device 214 can be heat set with the fixture to create the curvature in the plurality of members 220 illustrated in FIGS. 9A and 9B.

In some variants, the total length of the expansion device 214 can be about 1.000 inches. In some variants, the width of the plurality of members 220 can be about 0.021 inches. In some variants, the distance between the radial centers of curvature of opposing curved portions 250 of the proximal portion 216 and distal portion 218 can be about 0.750 inches. An aperture in the attachment portions 246 can be about 0.012 inches.

FIG. 9B illustrates a flat pattern for an expansion device 214 with three members 220. The flat pattern, including the example dimensions, and formation process can be similar to that described in reference to FIG. 9A.

FIG. 11 illustrates the cutting device 208 with the frame 209. As described herein, the frame 209 can include a plurality of struts 234 joined at connecting portions 258 to form a plurality of cells 236, which can have diamond shapes. The frame 209 can include the open proximal end 228, the open distal end 230, and the interior lumen 232 disposed between and connecting the open proximal end 228 and the open distal end 230. As illustrated the frame 209 can include the plurality of cutting elements 210, which can establish a cutting diameter of the cutting device 208. The cutting elements 210 can include an inward-projecting portion 254, that can curve radially inward from the tubular shape define by the struts 234 and connecting portions 258. The cutting elements 210 can include offset cutting portions 256 that can include the cutting tip and/or edge. The cutting portions 256 can project from the inward-projecting portions 254 in a proximal direction, which can include in an axial direction that is parallel to the central longitudinal axis of the frame 209. The cutting portions 256 can be offset but parallel to the planes of the portions of the cutting elements 210 distal of the inward-projecting portion 254. The cutting portions 256 can be offset in a radially inward direction relative to the tubular structure formed by the plurality of cells 236. The cutting elements 210 can include proximal attachment portions 252, which can include holes, to facilitate coupling (e.g., fastening, tying) the members 220 of the expansion device 214 to the cutting elements 210. The frame 209 can include a V-shaped gap 233 that is open in a proximal direction between adjacent cutting elements 210. The V-shaped gap 233 can be created by omitting proximal struts 234 of cells 236 disposed between adjacent cutting elements 210. The V-shaped gaps 233 can include axial lengths that are the same as the axial lengths of the diamond-shaped cells 236 of the frame 209. The V-shaped gaps 233 can help facilitate movement of the cutting elements 210 by the expansion device 214.

The frame 209, including the cutting elements 210, can be formed using a cylindrical structure disposed inside the frame 209 that includes at least two consistent diameters—an outer diameter for the tubular structure formed by the plurality of cells 236 and an inner diameter for the offset cutting portions 256—and a curved annular structure extending between and connecting the outer diameter and the inner diameter that can be used to form the inward-projecting portion 254 of the cutting elements 210. The frame 209 can be disposed around the cylindrical structure and formed therewith, which can include heat setting.

FIG. 11 illustrates a flat pattern for the frame 209 of the cutting device 208. The flat pattern can be cut from a sheet of material, such as Nitinol. To form the assembled frame 209, the flat pattern can be formed (e.g., rolled) into a tubular structure, which can include being formed (e.g., rolled) around the cylindrical structure described herein. Split connecting portions 258 can be coupled together, which can at least include welding and/or adhering, to hold the frame 209 in its tubular shape.

In some variants, the length of the cutting device 208 can be about 2.000 inches. The width of the cutting elements 210 can be about 0.039 inches. The width of the holes of the proximal attachment portions 252 can be about 0.012 inches. The width of each of the plurality of struts 234 can be about 0.010 inches. The width of the distal attachment portions 212 can be about 0.039 inches. The width of the holes of the distal attachment portions 212 can be about 0.012 inches.

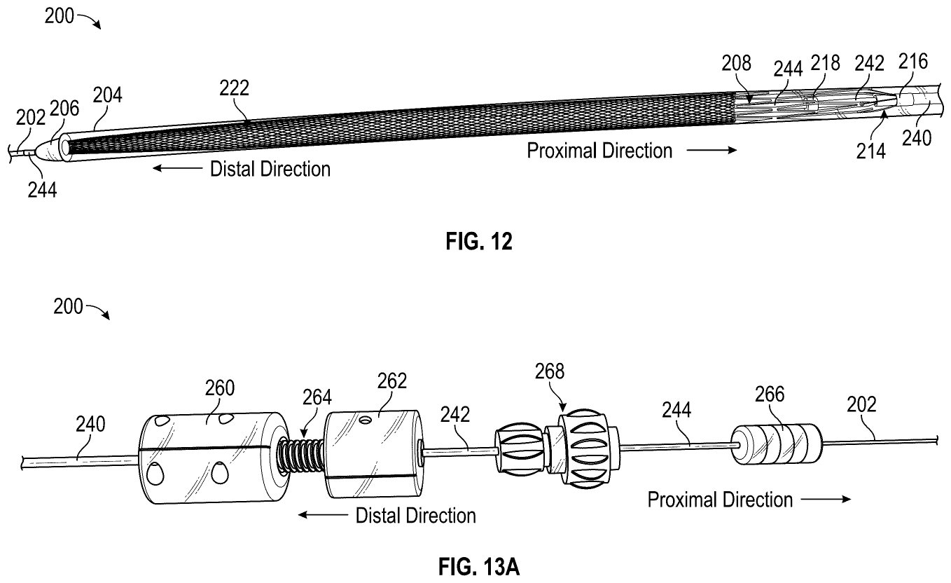

FIG. 12 illustrates the thrombectomy device 200 with the collection device 222, cutting device 208, and expansion device 214 in a collapsed configuration and disposed within a sheath 204. As shown, the tip 206 can be disposed at a distal end of the sheath 204, which can close the distal end of the sheath 204. The tip 206 can help dilate around the guide wire 202 as the thrombectomy device 200 is advanced along the guide wire 202 and through a thrombus 108 to position the thrombectomy device 200 distal of the thrombus 108.

In use, the thrombectomy device 200 can be advanced distally over the guide wire 202 to position the thrombectomy device 200 distal of the thrombus 108. The sheath 204 an be proximally retracted to uncover the collection device 222, cutting device 208 and expansion device 214. The expansion device 214 can expand radially outward to expand the cutting device 208, which expands the collection device 222. For example, the inner expander shaft 242 can be retracted proximally relative to the outer expander shaft 240 to move the distal portion 218 closer to the proximal portion 216 such that the plurality of members 220 move outward with the cutting elements 210 of the cutting device 208. With the collection device 222, cutting device 208, and expansion device 214 expanded, the thrombectomy device 200 can be proximally retracted to engage the cutting elements 210 with the thrombus 108. The expansion device 214 can hold the cutting elements 210 at a desired cutting diameter, which can include at the wall 118 of the vein 104 or radially inward thereof to core a thrombus 108 to restore blood flow therethrough. The expansion device 214 can provide sufficient hoop stress and/or compression force to maintain the cutting elements 210 at a desired cutting diameter. As described herein, portions of the cutting device 208 (e.g., open proximal end 228 and/or cutting elements 210) and/or expansion device 214 can be energized to ease cutting of the thrombus 108. In some variants, an energized element (e.g., conductor, wire) can be disposed at (e.g., wrapped around) portions of the cutting device 208 (e.g., open proximal end 228 and/or cutting elements 210) and/or expansion device 214 to ease cutting the thrombus. The cutting elements 210 can cut the thrombus 108, which can include coring the thrombus 108. The cut thrombus 108 can be moved into the open proximal end 228, through the interior lumen 232, out the open distal end 230 of the frame 209 of the cutting device 208 and into the interior 235 of the collection device 222 through the open proximal end 224 as the thrombectomy device 200 is proximally retracted. With the thrombus 108 in the collection device 222, the expansion device 214 can be collapsed to collapse the cutting device 208 and collection device 222, which can trap the thrombus 108 within the collection device 222. The expansion device 214 can be collapsed radially inward by distally advancing the inner expander shaft 242 relative to the outer expander shaft 240 to move the distal portion 218 away from the proximal portion 216 such that the plurality of members 220 move inward with the cutting elements 210 of the cutting device 208. The collapsed thrombectomy device 200 can be retraced proximally out of the patient with the trapped thrombus 108. In some variants, the relative positioning of the

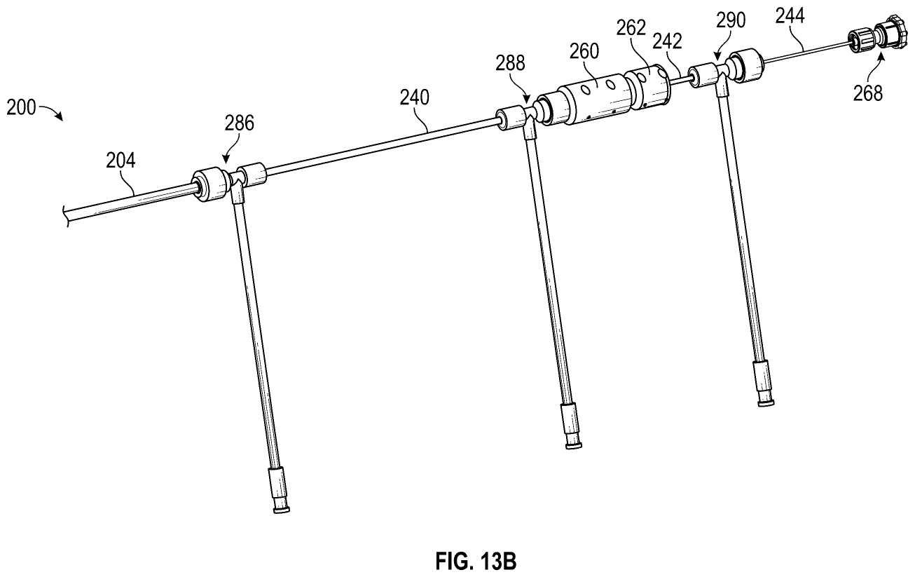

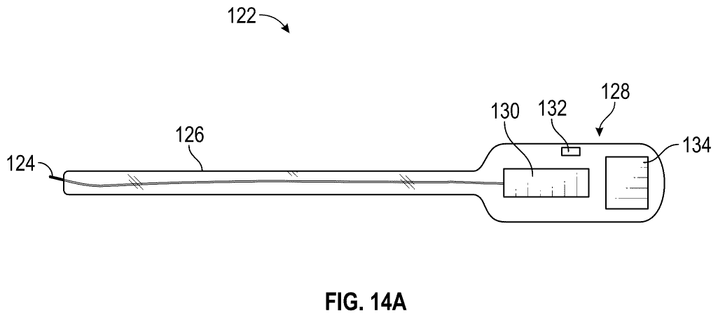

FIG. 13A illustrates an example proximal portion of the thrombectomy device 200 that can be disposed outside of the patient and used by a clinician to operate the thrombectomy device 200. The outer expander shaft 240 can include a handle 260. The inner expander shaft 242 can include a handle 262. The clinician can move the handle 262 relative to the handle 260 to move the inner expander shaft 242 relative to the outer expander shaft 240. In some variants, a threaded connection 264 can connect the handle 262 and handle 260 which can facilitate controlled movement of the handle 262 relative to the handle 260. For example, the clinician can rotate the handle 262 in a first direction which can move the handle 262 axially toward the handle 260 to distally advance the inner expander shaft 242 relative to the outer expander shaft 240 to collapse the expansion device 214. The clinician can rotate the handle 262 in a second direction, opposite the first, to move the handle 262 axially away from the handle 260 to proximally retract the inner expander shaft 242 relative to the outer expander shaft 240 to expand the expansion device 214. The thrombectomy device 200 can include a Tuohy Borst valve 268, which can be function as a lock, at the distal end of the inner expander shaft 242. The Tuohy Borst valve 268 can seal between the inner expander shaft 242 and inner shaft 244. The inner shaft 244 can include a handle 266. The guide wire 202 can be routed through the inner shaft 244. In some variants, the inner shaft 244 can be moved relative to the inner expander shaft 242 and outer expander shaft 240, which can move the tip 206 and/or collection device 222 relative to the inner expander shaft 242 and outer expander shaft 240. The clinician can distally advance the handle 260, handle 262, Tuohy Borst valve 268, and/or handle 266 with the outer expander shaft 240, inner expander shaft 242, and/or inner shaft 244 all together when distally advancing the thrombectomy device 200 over the guide wire 202. The clinician can proximally retract the handle 260, handle 262, Tuohy Borst valve 268, and/or handle 266 with the outer expander shaft 240, inner expander shaft 242, and/or inner shaft 244 all together when proximally retracting the thrombectomy device 200 over the guide wire 202.

FIG. 13B illustrates another example proximal portion of the thrombectomy device 200 that can be disposed outside of the patient and used by a clinician to operate the thrombectomy device 200. The sheath 204 can include a hemostasis valve 286 disposed at a proximal portion thereof that can include a port. The outer expander shaft 240 can include a hemostasis valve 288 disposed at a proximal portion thereof that can include a port. The handle 260 can be coupled to the outer expander shaft 240. The handle 262 can be coupled to the inner expander shaft 242. The inner expander shaft 242 can include a hemostasis valve 290 disposed at a proximal portion thereof that can include a port. The Tuohy Borst valve 268 can be disposed at a proximal portion of the inner shaft 244.

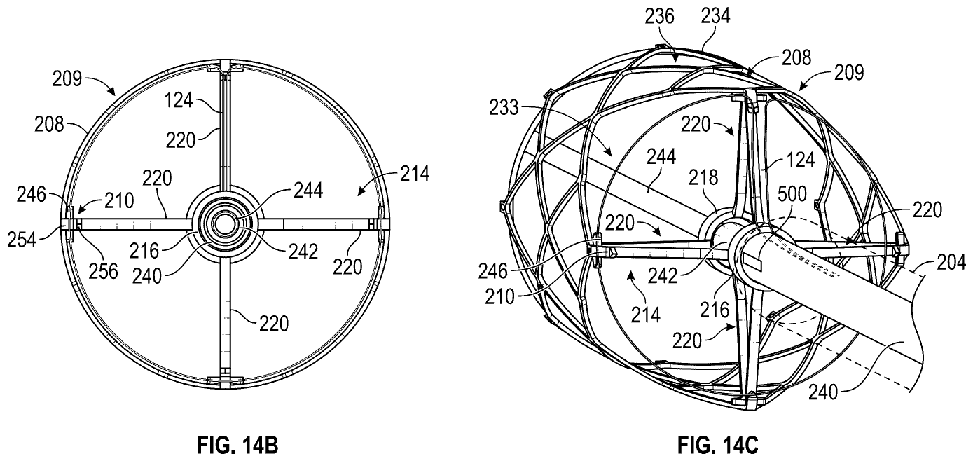

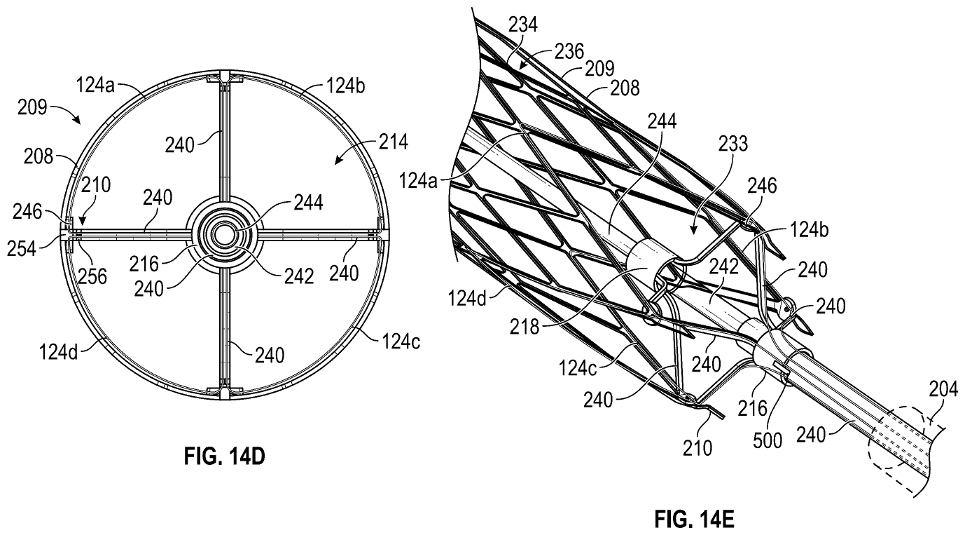

FIG. 14 illustrates a thermal device 122 (e.g., energy delivery device) that can be incorporated with (e.g., used with) the thrombectomy device 200. The thermal device 122 can provide heat to the thrombectomy device 200 to ease cutting through the thrombus. The heat can be applied to any portion of the thrombectomy device 200, which can at least include the collection device 222, expansion device 214, and/or cutting device 208 (e.g., cutting elements 210). The heat can heat the portion of the thrombectomy device 200.

The thermal device 122 can include a conductor 124 (e.g., wire, element, heated conductor). The conductor 124 can be at least solid or braided. The conductor 124 may include one or more materials. For example, the conductor 124 may include a metal, such as steel (e.g., stainless steel) and/or an alloy of nickel and titanium (e.g., Nitinol).

The thermal device 122 can include various software and hardware components to implement aspects of this disclosure, which may at least include a temperature modulation unit 130, controller 132, and/or power source 134 or at least an interface to receive energy from a power source. The conductor 124 may be operatively connected to a temperature modulation unit 130. The temperature modulation unit 130 may adjust the temperature of the conductor 124 (e.g., electrode, heating element, heater, etc.). For example, the temperature modulation unit 130 may raise the temperature (e.g., heat) of the conductor 124. The temperature modulation unit 130 may heat the conductor 124 directly or indirectly with one or more energy sources, which may at least include heat, radio frequency, laser, electricity (e.g., current, direct current, alternative current), resistive heating, inductive heating, nuclear, heated liquid, and/or others. For example, the temperature modulation unit 130 may apply a current of electricity to the conductor 124, raising the temperature of the conductor 124. In some variants, the temperature modulation unit 130 may lower the temperature (e.g., cool) the conductor 124. The temperature of the wire 124 may be automatically modulated based on monitored conditions (e.g., sensed with a temperature sensor) in a blood vessel and/or at the thrombus. The temperature of the conductor 124 may be adjusted to accommodate convection losses. The temperature of the conductor 124 may be controlled by a clinician. The controller 132 may be operatively connected with the temperature modulation unit 130 to perform the temperature control described herein by way of the temperature modulation unit 130. The conductor 124 can, in some variants, be heated to any temperature or range of temperatures between 10 and 300 degrees Celsius. In some variants, the conductor 124 can be heated to any temperature or range of temperatures between 10 and 200 degrees Celsius. In some variants, the conductor 124 can be heated to any temperature or range of temperatures between 10 and 200 degrees Celsius. In some variants, the conductor 124 can be heated to any temperature or range of temperatures between 10 and 50 degrees Celsius. In some variants, the conductor 124 can be heated to any temperature below 10 degrees Celsius.

The power source 134 may be a battery, which may include a rechargeable battery and/or disposable one-time-use battery. The power source 134 may power the thermal device 122, which can include the controller 132 and/or temperature modulation unit 130. The power source 134 may supply the energy to adjust the temperature (e.g., heat) the conductor 124. In some variants, the thermal thrombectomy system 122 may be operatively connected to an external power source.

The thermal device 122 may also include memory, communication interface(s) (wired or wireless), user interfaces (e.g., button(s), dial(s), switch(es), display(s), touchpad(s), touchscreen(s), knob(s), trigger(s), indicator(s), gauge(s), and/or slider(s)), etc. to implement aspects of the disclosure. The temperature modulation unit 130, controller 132, power source 134, and/or other components of the thermal thrombectomy system 122 may be housed in a housing 128, which may be a handle.

The thermal device 122 may include insulation 126, which may also be referred to as a covering. The insulation 126 may insulate the conductor 124 from the anatomy of the patient, such as the blood vessel. The insulation 126 may be disposed over a proximal portion of the conductor 124, which may include being disposed over the entirety of the conductor 124 except a distal portion. The conductor 124, in some variants, may be deployed from and retracted into the insulation 126. In some variants, the insulation 126 may be an insulating coating disposed on the conductor 124.

The conductor 124 can be disposed around features of the thrombectomy device 200 described herein to provide energy (e.g., heat) to ease penetrating, passing, and/or cutting the thrombectomy device 200. In some variants, the conductor 124 delivers energy (e.g., heat) to one or more features of the thrombectomy device 200 to energize the one or more features themselves. The conductor 124 can be heated, which can include being heated with electrical energy such as direct current. The conductor 124 can include a first portion carrying electrical energy distally and a second portion carrying electrical energy proximally. The conductor 124 can form a loop that is routed around features of the thrombectomy device 200. In addition to providing heat, the structure of the conductor 124 can cut the thrombus.

The conductor 124 can be disposed on various portions of the thrombectomy device 200. For example, the conductor 124 can be disposed on the collection device 222, cutting device 208 (e.g., cutting elements 210), expansion device 214, and/or outer expander shaft 240. The conductor 124 can be heated as described herein to provide heat for cutting. Proximal portions of the conductor 124 can be covered with insulation 126, which can help protect anatomy. In some variants, the conductor 124 can include one or more conductor 124 (e.g., one, two, three, four, five, six, seven, eight, nine, ten, eleven, twelve or more conductors 124). With multiple conductors 124, the conductors 124 can be separately routed to different portions of the thrombectomy device 200.

FIGS. 14B and 14C illustrate the thrombectomy device 200 with a conductor 124. As illustrated, the conductor 124 can be routed through the sheath 204, which can include being disposed on an exterior of the outer expander shaft 240. The conductor 124 can be coupled to the cutting elements 210, which can include being coupled to the cutting portions 256, inward-projecting portion 254, and/or other portions. The conductor 124 can be disposed at the proximal open end of the cutting device 208. The conductor 124 can span between the cutting elements 210. The conductor 124 can form a loop having a circular shape. The conductor 124 can follow a cutting diameter of the one or more cutting elements 210. The conductor 124 can be routed distally along the outer expander shaft 240. The conductor 124 can be routed along a proximal portion of one of the plurality of members 220 of the expansion device 214 to one of the cutting elements 210. The conductor 124 can follow a cutting diameter of the cutting elements 210. The conductor 124 can form a loop that extends between the cutting elements 210. The conductor 124 can form a circle. The conductor 124 can be coupled to the cutting elements 210. The conductor 124 can be routed proximally back along the outer expander shaft 240. The conductor 124 can be routed proximally along the proximal portion of one of the plurality of members 220, which can be the same member 220 on which the conductor 124 was routed distally. The conductor 124 can be coupled to any of the features shown in FIGS. 14B and 14C. As described herein, the conductor 124 can be heated, which can include being heated with electrical energy. The heat from the conductor 124 can ease cutting the thrombus. The structure of the conductor 124 can also cut the thrombus. For example, the portions of the conductor 124 suspended between cutting elements 210 can cut the thrombus. The thrombectomy device 200 can include a temperature sensor 500 (e.g., thermistor, thermocouple, etc.) that can sense temperatures, which can include sensing temperatures proximate the conductor 124, expansion device 214, outer expander shaft 240, inner expander shaft 242, cutting elements 210, frame 209, and/or anatomy of the patient. The temperature of the conductor 124 can be controlled based on temperatures sensed by the temperature sensor 500, which can include automatic control by the thermal device 122 and/or control by a clinician.

FIGS. 14D and 14E illustrate the thrombectomy device 200 with a plurality of conductors 124, which can include conductors 124a, 124b, 124c, and 124d. Each of the conductors 124 can be routed along a different path. The conductors 124 can be routed distally through the sheath 204, which can include along an exterior of the outer expander shaft 240. Each of the conductors 124 can include a first portion that carries electrical energy distally and a second portion that carries electrical energy proximally. Each of the conductors 124 can form a loop. Each of the conductors 124 can be routed distally along the outer expander shaft 240, distally along a proximal portion of one of the plurality of members 220, to one of the cutting elements 210, along the proximal portions of the frame 209 between adjacent cutting elements 210 (e.g., along the plurality of struts 234 forming the V-shaped gap 233), to an adjacent one of the cutting elements 210, proximally along a proximal portion of an adjacent one of the plurality of plurality of members 220, and/or proximally along the outer expander shaft 240. Each of the conductors 124 can form a loop. As described herein, the conductors 124 can be heated, which can include being heated with electrical energy. The heat from the conductors 124 can ease cutting the thrombus. The structure of the conductors 124 can also cut the thrombus.

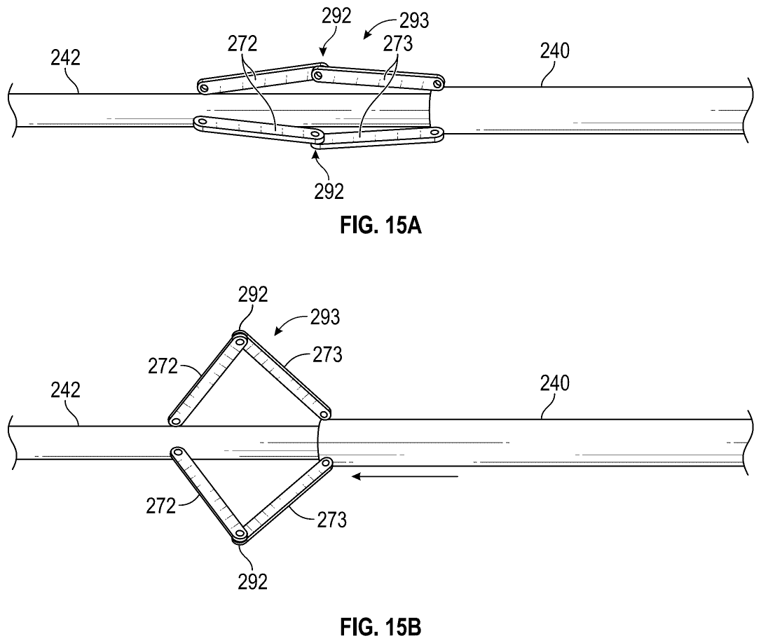

FIGS. 15A and 15B illustrate an expansion device 293 that can be used with the thrombectomy device 200. The expansion device 293 can include distal links 272 and/or proximal links 273. The distal links 272 can be coupled (e.g., rotatably coupled) to the inner expander shaft 242. The proximal links 273 can be coupled (e.g., rotatably coupled) to the outer expander shaft 240. The distal links 272 and proximal links 273 can be coupled together by joints 292. The distal links 272 and proximal links 273 can pivot with respect to each other about the joints 292. The cutting elements 210 of the cutting device 208 can be coupled to the joint 292. As illustrated in FIG. 15A, the expansion device 293 can be collapsed with the distal advancement of the inner expander shaft 242 relative to the outer expander shaft 240. The distal links 272 and proximal links 273 can rotate inward (e.g., collapse inward), which can collapse the cutting device 208 coupled to the joints 292. As illustrated in FIG. 15B, the expansion device 293 can be expanded with the proximal retraction of the inner expander shaft 242 relative to the outer expander shaft 240. The distal links 272 and proximal links 273 can rotate outward (e.g., expand outward), which can expand the cutting device 208 coupled to the joints 292. The expansion device 293 can be used to expand or collapse the cutting device 208 in the methods described herein.

FIGS. 16A and 16B illustrate an expansion device 270 that can be used with the thrombectomy device 200. The expansion device 270 can include the distal links 272 and/or wires 278. The distal links 272 can be rotatably coupled to the inner expander shaft 242. The wires 278 can be coupled to the outer expander shaft 240. The distal links 272 and wires 278 can be coupled (e.g., rotatably coupled) together at the proximal attachment portions 274 of the distal links 272. The cutting elements 210 of the cutting device 208 can be coupled to the proximal attachment portions 274. As illustrated in FIG. 16A, the expansion device 270 can be collapsed with the distal advancement of the inner expander shaft 242 relative to the outer expander shaft 240. The distal links 272 and wires 278 can rotate inward (e.g., collapse inward), which can collapse the cutting device 208 coupled to the proximal attachment portions 274. As illustrated in FIG. 16B, the expansion device 270 can be expanded with the proximal retraction of the inner expander shaft 242 relative to the outer expander shaft 240. The distal links 272 and wires 278 can rotate outward (e.g., expand outward), which can expand the cutting device 208 coupled to the proximal attachment portions 274. The expansion device 270 can be used to expand or collapse the cutting device 208 in the methods described herein.

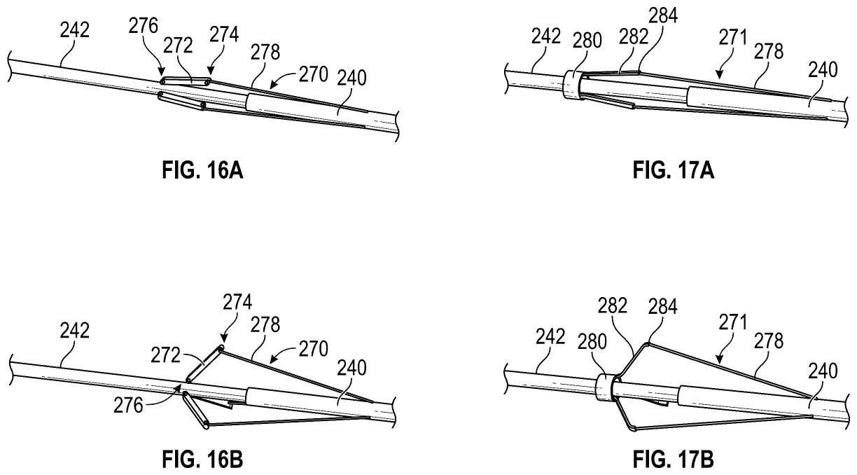

FIGS. 17A and 17B illustrate an expansion device 271 that can be used with the thrombectomy device 200. The expansion device 271 can include members 282 and/or wires 278. The members 282 can coupled to the inner expander shaft 242 with a distal portion 280, which can be an annular structure disposed on the inner expander shaft 242. One end of the members 282 can be coupled to the distal portion 280. The wires 278 can be coupled to the outer expander shaft 240. The members 282 and wires 278 can be coupled together at the end portions 284 (e.g., proximal end portions) of the members 282. The cutting elements 210 of the cutting device 208 can be coupled to the end portions 284. As illustrated in FIG. 17A, the expansion device 271 can be collapsed with the distal advancement of the inner expander shaft 242 relative to the outer expander shaft 240. The members 282 and wires 278 can rotate inward (e.g., collapse inward), which can collapse the cutting device 208 coupled to the end portions 284. As illustrated in FIG. 17B, the expansion device 271 can be expanded with the proximal retraction of the inner expander shaft 242 relative to the outer expander shaft 240. The members 282 and wires 278 can rotate outward (e.g., expand outward), which can expand the cutting device 208 coupled to the end portions 284. The expansion device 271 can be used to expand or collapse the cutting device 208 in the methods described herein.

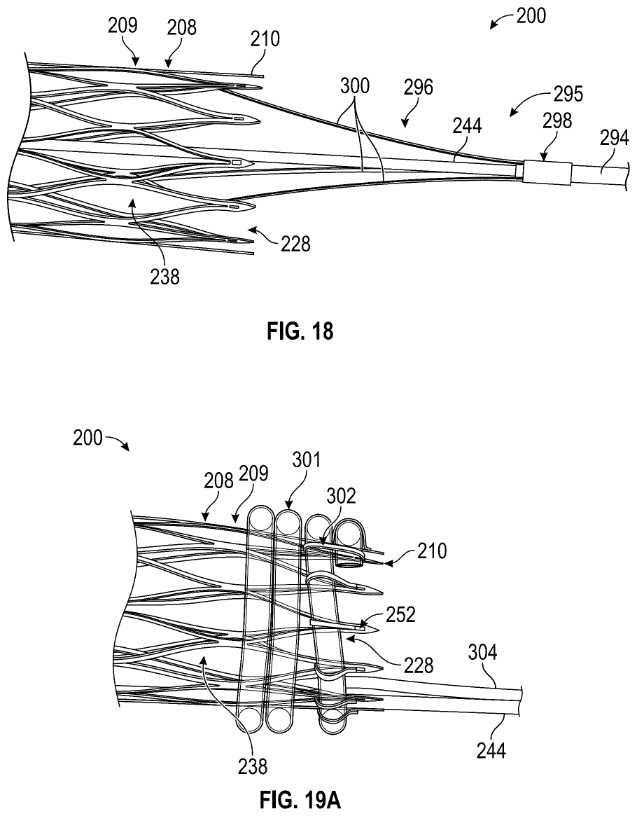

FIG. 18 illustrates an expansion device 295 that can be used with the thrombectomy device 200. The expansion device 295 can include a proximal portion 298, which can be an annular structure. The proximal portion 298 can be coupled to (e.g., disposed on) the outer shaft 294, which can be one of the other shafts described herein. The expansion device 295 can include a plurality of members 300. The plurality of members 300 can curve distally away from the proximal portion 298 and radially outward to coupled to the frame 209 of the cutting device 208. With the distal advancement of the inner shaft 244 relative to the outer shaft 294, the plurality of members 300 can rotate inward (e.g., collapse), which can collapse the cutting device 208. With the proximal retraction of the inner shaft 244 relative to the outer shaft 294, the plurality of members 300 can rotate outward (e.g., expand), which can expand the cutting device 208. The expansion device 295 can be used to expand or collapse the cutting device 208 in the methods described herein.

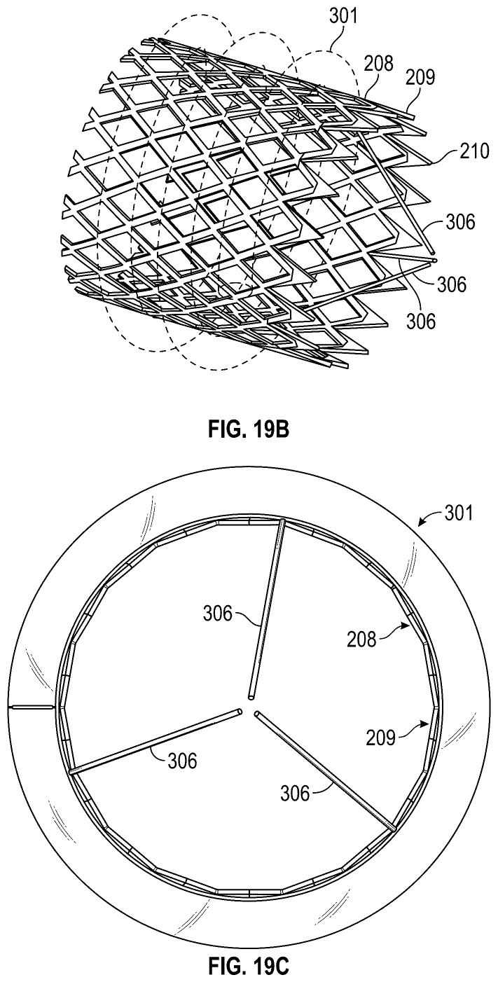

As illustrated in FIG. 19A, the thrombectomy device 200 can utilize a balloon 301 to expand and collapse the cutting device 208. The balloon 301 can be coiled around the proximal portion of the frame 209 of the cutting device 208. The balloon 301 can be coupled to the cutting elements 210, which can include being coupled to the cutting elements 210 with fasteners 302 (e.g., bands, ties, filament). The fasteners 302 can extend around the balloon 301 and through the proximal attachment portions 252. The thrombectomy device 200 can include an inflation tube 304 that can deliver a medium (e.g., gas, fluid) to the balloon 301 for inflation and/or carry the medium away from the balloon 301 for deflation. The inner shaft 244 can be disposed off-center of the central longitudinal axis of the frame 209 of the cutting device 208. When the balloon 301 is inflated, the balloon 301 can expand radially outward with the cutting device 208 coupled to the balloon 301. When the balloon 301 is deflated, the balloon 301 can collapse radially inward with the cutting device 208 coupled to the balloon 301. The balloon 301 can be used to expand or collapse the cutting device 208 in the methods described herein.

FIG. 19B illustrates a cutting device 208 with a coiled balloon 301 to expand or collapse the cutting device 208 as described herein. The cutting device 208 can include members 306 (e.g., three members 306) that extend radially inward from the frame 209 of the cutting device 208 to center the cutting device 208 about a shaft, such as the inner shaft 244. FIG. 19C illustrates an axial view of the cutting device 208 with the members 306 that extend radially inward.

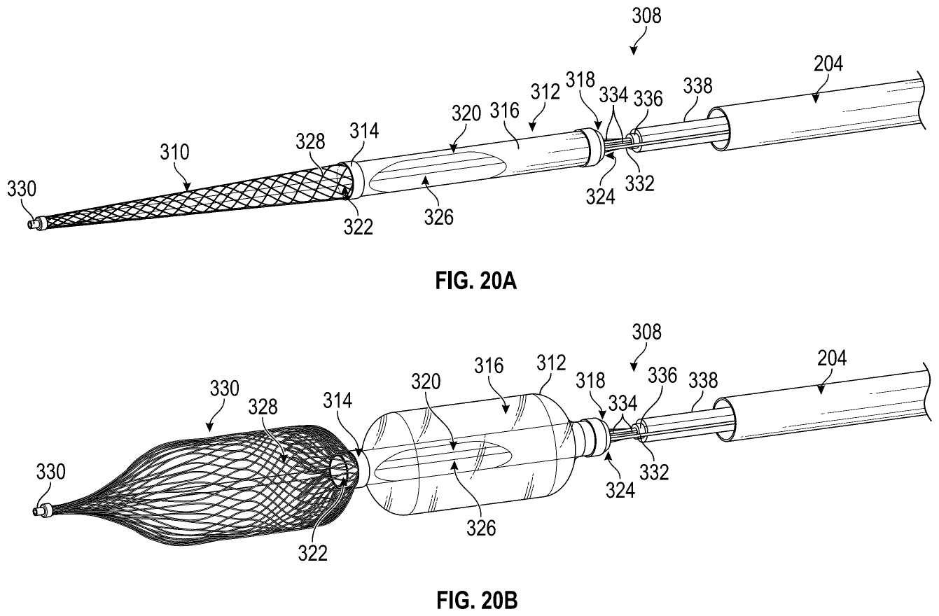

FIG. 20A illustrates a thrombectomy device 308, which can include any of the features of the other thrombectomy devices described herein. The thrombectomy device 308 can include a collection device 310, which can include any of the features of the collection device 222. The collection device 310 can include a closed distal end and an open proximal end. The collection device 310 can include an interior 328 accessible through the open proximal end. In some variants, the collection device 310 can be self-expanding when unsheathed from the sheath 204. In some variants, the collection device 310 can be coupled to the inner shaft 330 such that proximal retraction of the inner shaft 330 causes the collection device 310 to expand

The thrombectomy device 308 can include a cutting device 312 (e.g., coring device). The device 312 can include a cylindrical structure 314. The cylindrical structure 314 can include a distal opening 322, a proximal opening 324, and an interior 326 extending between the proximal opening 324 and the distal opening 322. The distal opening 322 can be coupled to the open proximal end of the collection device 310. The cylindrical structure 314 can include a cutting element 318, which can be a cutting ring. The cutting element 318 can be disposed at the proximal opening 324 of the cylindrical structure 314. The cutting element 318 can include a cutting ring that is fixed in size and/or shape. In some variants, the cutting element 318 can include a cutting ring that is expandable. In some variants, the cylindrical structure 314 can be replaced with a spring that can provide a working lumen. The spring can enable the thrombectomy device 308 to be further collapsed for deliver through smaller sized sheath 204. A proximal end of the cutting spring can include the cutting element 318, which can be a cutting ring.

The cutting device 312 can include a balloon 316. In some variants, the balloon 316 can be non-compliant. The balloon 316 can be disposed on the cylindrical structure 314. The balloon 316 can include an annular shape. The thrombectomy device 308 can include an inflation tube 332 to deliver a medium (e.g., gas, fluid) to the balloon 316. The inflation tube 332 can routed through the interior 326 of the cylindrical structure 314 to deliver the medium to the balloon 316 through a side opening 320 in the cylindrical structure 314. The inflated balloon 316 can engage the wall 118 of the vein 104, which can center the proximal opening 324 and/or cutting element 318 for coring the thrombus 108 during proximal retraction. The balloon 316 can be inflated periodically to assure balloon 316 is expanding to maximum profile of wall 118.

The thrombectomy device 308 can include an inner shaft 330 that can be disposed through the cylindrical structure 314 and collection device 310. The thrombectomy device 308 can include an outer shaft 338 through which the inner shaft 330 can extend. The outer shaft 338 can extend through the sheath 204. The inflation tube 332 can extend through the sheath 204. The thrombectomy device 308 can include a proximal attachment portion 336, which can be an annular structure, that can be coupled to the outer shaft 338 or inner shaft 330. Members 334 can extend distally from the proximal attachment portion 336 and couple with the cylindrical structure 314 and/or collection device 310.

FIG. 20B illustrates the thrombectomy device 308 with the collection device 310 expanded and the balloon 316 inflated.

In use, the collapsed thrombectomy device 308 can be disposed within the sheath 204 and advanced distally over a guide wire 202 disposed through the inner shaft 330 to a position distal of a thrombus 108. The sheath 204 can be proximally retracted. Medium can be delivered to the balloon 316 to expand the balloon 316. The collection device 310 can be self-expanding or manually expanded by way of translating one or more shafts, such as the inner shaft 330 and/or outer shaft 338. With the balloon 316 expanded, the proximal opening 324 and/or cutting element 318 can be centered relative to the wall 118 of the vein 104. The thrombectomy device 308 can be retracted proximally to engage the cutting element 318 with the thrombus 108 which can cut a portion (e.g., core) of the thrombus 108. The cut thrombus 108 can move into the proximal opening 324, through the interior 326, and out the distal opening 322 into the interior of the collection device 310 as the thrombectomy device 308 is retracted proximally. The balloon 316 and/or collection device 310 can be collapsed, which can include deflating the balloon 316. The thrombectomy device 308 with the trapped thrombus can be retracted proximally out of the patient.

In some variants, the cutting element 318 can include a diameter of 5-6 mm. In some variants, the sheath 204 can be 17 Fr. In some variants, the balloon 316 can be 10-15 mm.

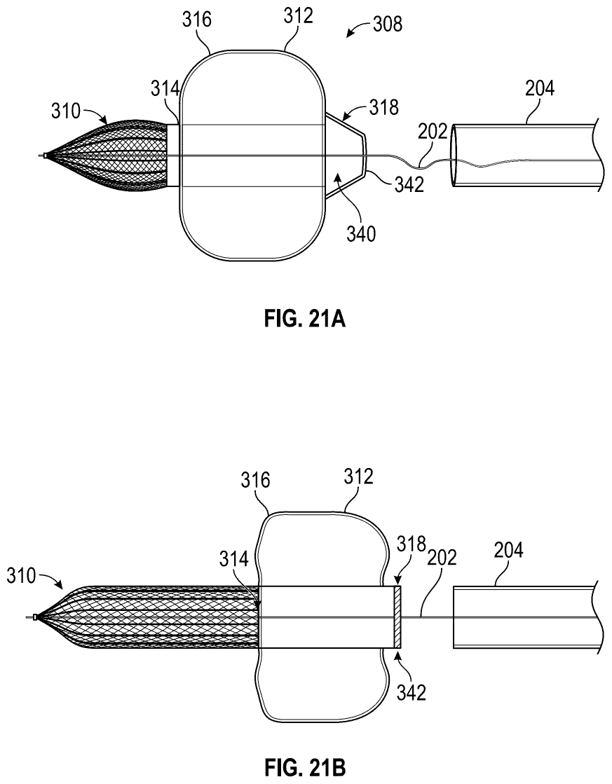

FIG. 21A schematically illustrates the thrombectomy device 308 with a cutting element 318 that includes an open truncated cone 340, which can also be referred to as an open conical frustrum. The proximal portion of the open truncated cone 340 can include a cutting ring 342, which can be disposed at the proximal opening into the open truncated cone 340. A thrombus 108 cut by the cutting ring 342 of the cutting element 318 can move through the cylindrical structure 314 to an interior of the collection device 310. In some variants, the guide wire 202 can be anchored proximate the cutting ring 342 and/or proximate the distal end of the cylindrical structure 314. The cutting ring 342 can include a diameter between 2-20 mm, which can include 0.5 or 1 mm increments. The angle of the walls of the open truncated cone 340 can be about 10-90 degrees (e.g., 30 degrees). The diameter of the inner lumen of the cylindrical structure 314 can match that of the cutting ring 342. The balloon 316 can be inflated up to 25 mm. The collection device 310 can be less than six inches, about six inches, or more than six inches in length.

FIG. 21B schematically illustrates the thrombectomy device 308 with a cutting element 318 that includes a cutting ring 342. The cutting element 318 can be disposed at the proximal opening into the cylindrical structure 314, which can guide cut thrombus 108 into the collection device 310.

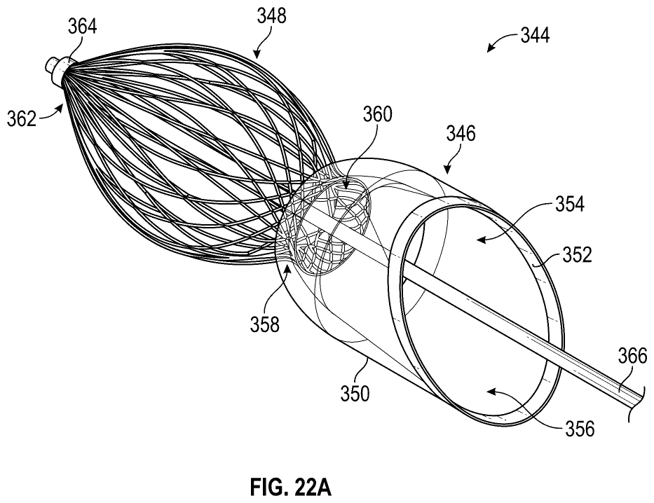

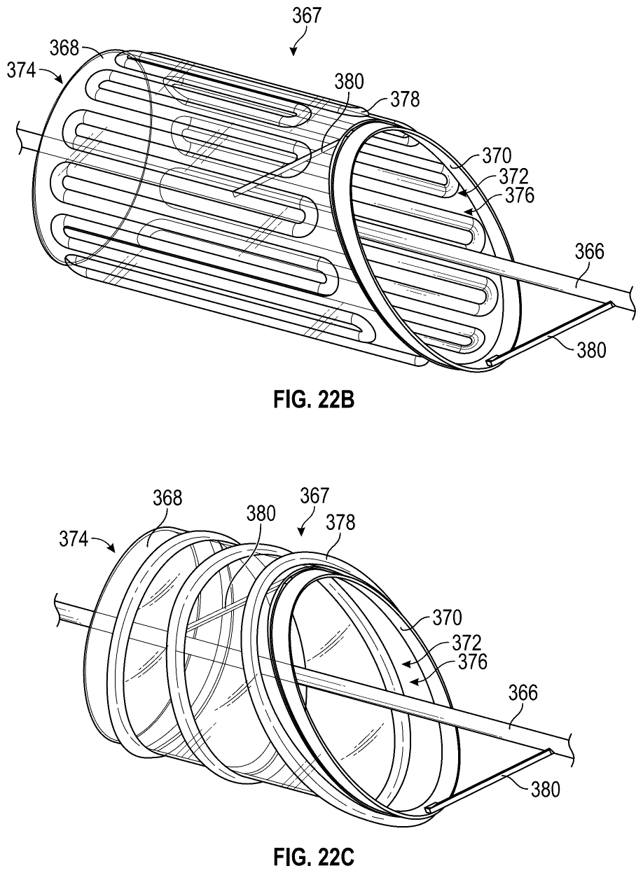

FIG. 22A illustrates a thrombectomy device 344, which can include any of the features of the other thrombectomy devices described herein. The thrombectomy device 344 can include a collection device 348 (e.g., basket, bag) and/or a cutting device 346 (e.g., coring device). The collection device 348 can include a tapered distal end 362. The tapered distal end 362 can be closed. The tapered distal end 362 can include a tip 364. The tip 364 can hold the filaments (e.g., wires) of the collection device 348. The collection device 348 can include a proximal open mouth 360 providing access into an interior of the collection device 348. In some variants, the collection device 348 can be self-expanding.

The cutting device 346 can include a balloon 350, which can have an annular shape. The balloon 350 can include an open distal end 358 that can be coupled to the proximal open mouth 360 of the collection device 348. The balloon 350 can include a proximal open end 354 proving access into an interior lumen 356 of the cutting device 346 (e.g., balloon 350). The interior lumen 356 can connect the proximal open end 354 and open distal end 358. The cutting device 346 can include a cutting ring 352, which can be angled (e.g., having a slope when viewed from the side). The thrombectomy device 344 can be disposed on an inner shaft 366.

In use, the thrombectomy device 344 can be routed to distal of a thrombus 108 and unsheathed. The balloon 350 can expand the cutting device 346. The collection device 348 can self-expand. The expansion of the balloon 350 can expand the cutting ring 352 disposed at a proximal open end 354 of the balloon 350. The thrombectomy device 344 with the expanded cutting device 346 and collection device 348 can be proximally retracted into a thrombus 108 to cut the thrombus 108. The cut thrombus can move into the proximal open end 354, through the interior lumen 356, and out the open distal end 358 and into the interior of the collection device 348 by way of the proximal open mouth 360. The cutting device 346 can be collapsed by deflating the balloon 350.

FIG. 22B illustrates a cutting device 367 (e.g., coring device) that can be similar to the cutting device 346. For example, the cutting device 367 can include a balloon 368, which can have an annular shape. The balloon 368 can include an open proximal end 372, an open distal end 374, and an interior lumen 376 extending between the open proximal end 372 and the open distal end 374. The cutting device 367 can include a cutting ring 370 at the open proximal end 372, which can be angled (e.g., sloped when viewed from the side). An inner shaft 366 can extend through the interior lumen 376 of the balloon 368. Members 380 (e.g., supports, struts, braces) can extend from the cutting ring 370 to the inner shaft 366. The members 380 can maintain the positioning of the cutting ring 370 during cutting and/or guide positioning when the balloon 368 is being expanded or collapsed. The balloon 368 can include an inflation channel 378 that can follow a serpentine path circumferentially around the balloon 368. The serpentine inflation channel 378 can provide rigidity to the balloon 368 when inflated.

FIG. 22C illustrates the cutting device 367 but with the balloon 368 having a coiled inflation channel 378 around the circumference. The coiled inflation channel 378 can provide rigidity to the balloon 368 when inflated.

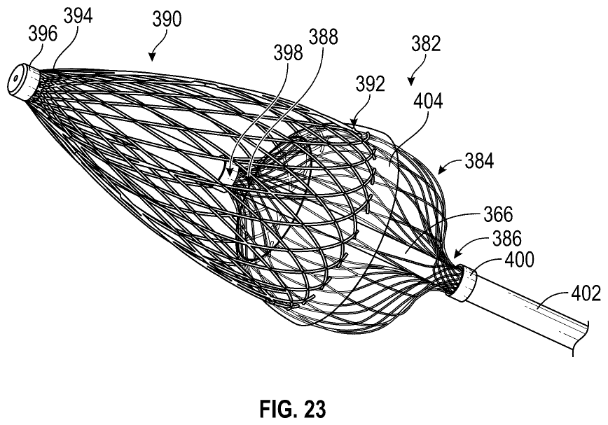

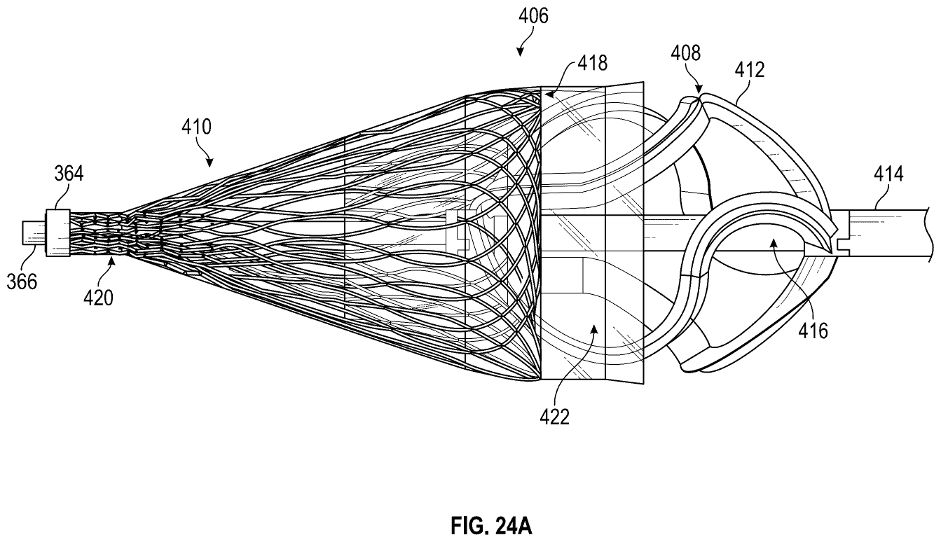

FIG. 23 illustrates a thrombectomy device 382, which can include any features of the other thrombectomy devices described herein. The thrombectomy device 382 can include a collection device 390 (e.g., mesh basket) and a cutting device 384, which can be self-expanding when unsheathed. The collection device 390 can include a closed distal end 394 and an open proximal end 392 providing access into an interior of the collection device 390. The collection device 390 can include a distal tip 396, which can be disposed at the closed distal end 394. The distal tip 396 can be disposed on a distal end of a inner shaft 366 passing through the thrombectomy device 382.

The cutting device 384 can include a mesh cutting basket that can have a ball-like shape. The cutting device 384 can include a closed proximal end 386, which can be tapered, and a closed distal end 388, which can be tapered. The distal end 388 can be secured to the inner shaft 366 with a distal attachment portion 398. The closed proximal end 386 can be secured to the outer shaft 402, which can receive the inner shaft 366 therethrough, with a proximal attachment portion 400. A sleeve 404, which can have an annular shape that is bowl-like, can be disposed between the cutting device 384 and the collection device 390. In use, the thrombectomy device 382 can be disposed distally of a thrombus and unsheathed to expand. The thrombectomy device 382 can be proximally retracted to cut the thrombus with the cutting device 384. The cut thrombus 108 can move through the sleeve 404 and into the collection device 390 by way of the open proximal end 392 for removal.

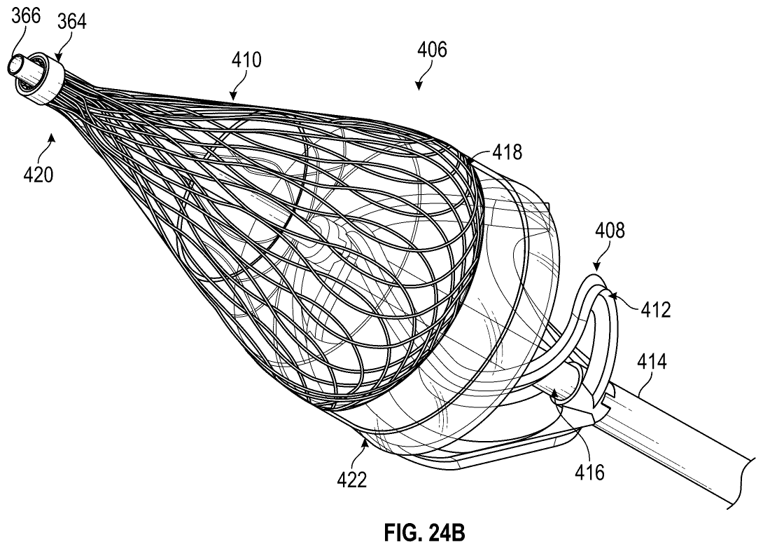

FIGS. 24A and 24B illustrate a thrombectomy device 406, which can include any of the features of the other thrombectomy devices described herein. The thrombectomy device 406 can include a collection device 410 (e.g., mesh basket) and/or a cutting device 408. The collection device 410 can include a closed distal end 420 and an open proximal end 418 providing access into an interior of the collection device 410. A tip 364 can be disposed at the closed distal end 420.

The cutting device 408 can include a plurality of cutting blades 412. The cutting blades 412 can be twisted. The cutting blades 412 can curve around a central longitudinal axis of the thrombectomy device 406. The cutting device 408 can be disposed on an outer main shaft 414. An inner main shaft 416 can be disposed through the cutting device 408, which can include radially inward of the cutting device 408. The inner shaft 366 can pass through the inner main shaft 416 and outer main shaft 414. In some variants, the cutting device 408 can collapse or expand by way of axial and/or rotational movement of one or more of the shafts illustrated in FIG. 24A. A sleeve 422, which can have an annular shape that is bowl-like, can be disposed between the cutting device 408 and the collection device 410. In use, the thrombectomy device 406 can be deployed distal of a thrombus 108 and retracted proximally to engage the cutting device 408 with the thrombus 108 for cutting. In some variants, the thrombectomy device 406 can be deployed between two balloons isolating the thrombus 108. In some variants, the cutting device 408 can be rotated to cut up the thrombus 108, which can include macerating the thrombus 108. The cut up thrombus 108 can enter the collection device 410 through the sleeve 422 and passage through the open proximal end 418.

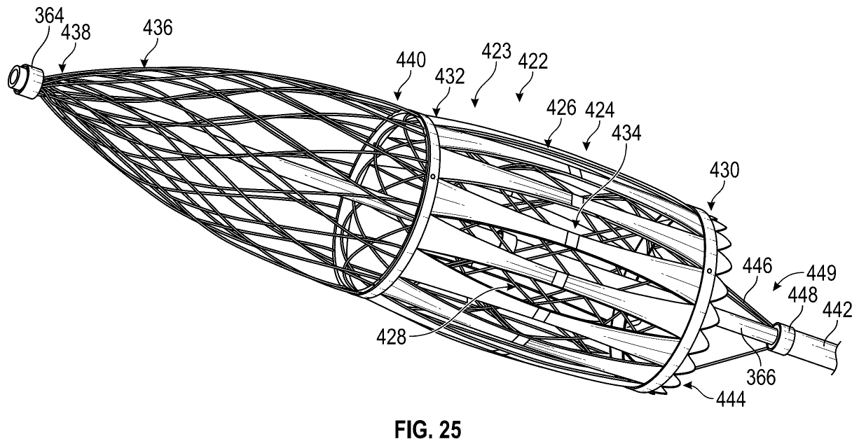

FIG. 25 illustrates a thrombectomy device 423, which can include any of the thrombectomy devices described herein. The thrombectomy device 423 can include a collection device 436 with a closed distal end 438 and open proximal end 440 providing access into an interior. The collection device 436 can include a tip 364. The tip 364 can be disposed on a inner shaft 366 disposed through the thrombectomy device 423.

The thrombectomy device 423 can include a cutting device 424 with a frame 426 and an inner mesh 428, which can help the frame 426 to expand. The frame 426 can include a distal end 432 coupled to the open proximal end 440 of the collection device 436 and an proximal end 430 providing access into an interior lumen 434 extending between and connecting the open proximal end 430 and open distal end 432. The frame 426 can include cutting elements 444 at the open proximal end 440, which can include being circumferentially distributed about the open proximal end 440.

The thrombectomy device 423 can include an expansion mechanism 449. The expansion mechanism 449 can include a proximal attachment portion 448, which can be an annular structure, coupled to an outer shaft 442. The expansion mechanism 449 can include a plurality of members 446 that can extend distally and radially outward from the proximal attachment portion 448 to the frame 426 of the cutting device 424. In some variants, with relative axial movement between the inner shaft 366 and the outer shaft 442, the cutting device 424 can be collapsed or expanded.

In use, the thrombectomy device 423 can be disposed distal of a thrombus 108, expanded, and proximally retracted to engage the cutting elements 444 with the thrombus 108 to cut the thrombus 108. The cut thrombus 108 can move into the proximal end 430, through the interior lumen 434, and out the distal end 432 of the cutting device 424 and into the collection device 436 by way of the open proximal end 440 for removal.

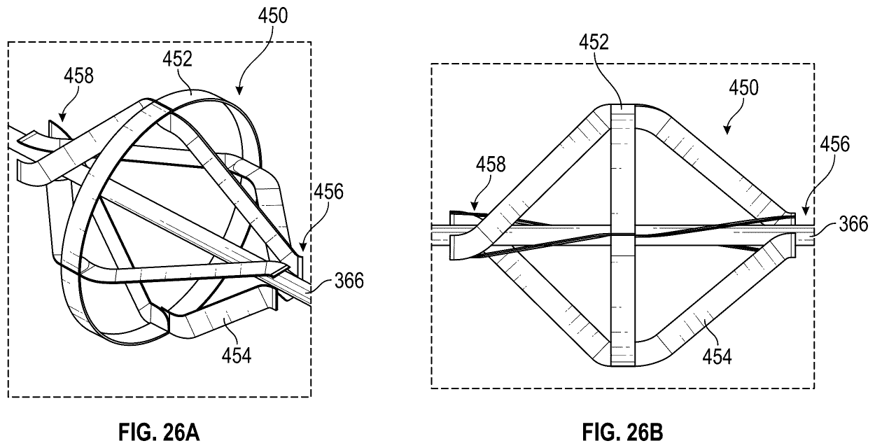

FIGS. 26A and 26B illustrate a cutting device 450 that can be used with a thrombectomy device to cut a thrombus 108. The cutting device 450 can include a cutting ring 452. The cutting device 450 can include a distal end 456 and proximal end 458. The cutting device 450 can include a plurality of cutting struts 454 that can extend from the distal end 456, which can be coupled to the inner shaft 366, to the cutting ring 452 and the proximal end 458, which can be coupled to the inner shaft 366, to the cutting ring 452. The struts 454 can include twists and/or slopes.

The actions, steps, methods, etc. described herein can be performed by a clinician (e.g. surgeon) and/or robot.

The collection devices described herein can include tubular structures. The collection devices can include open proximal ends and closed distal ends. The collection devices can be tapered. The collection devices can include meshed structures, which can include fine mesh. The meshed structures can include a weave of filaments. The collection devices can include bags and/or baskets.

In some variants, the thrombectomy devices described herein can be used with a variety of cutting ring sizes. The thrombectomy devices described herein can, in some variants, be collapsed back into a delivery sheath for multiple passes on a thrombus if necessary. Cutting rings can be sharp, serrated, energized, heated, and/or cooled. Cutting rings can be sharp, semi-circle, and/or spoked.

In some variants, the sheaths described herein can be 9-10 Fr. In some variants, the sheath described herein can be 7-8 Fr. In some variants, the guide wires described herein can be 0.035 inches. In some variants, one or more features described herein can self-expand, which can include cutting devices, frames, collection devices (e.g., bags, baskets), and/or other features. For example, one or more features can include Nitinol that is set to self-expand.

The thrombectomy devices described herein can at least be used in sub-acute and chronic occlusions for post-thrombotic syndrome, in-stent restenosis, AV fistula, etc. The shafts described herein can include internal lumens. The shafts described herein can be tubes.

### Terminology

Although the systems and methods have been disclosed in the context of certain embodiments and examples, it will be understood by those skilled in the art that the systems and methods extend beyond the specifically disclosed embodiments to other alternative embodiments and/or uses of the embodiments and certain modifications and equivalents thereof. Various features and aspects of the disclosed embodiments can be combined with or substituted for one another in order to form varying modes of the systems and methods. The scope of this disclosure should not be limited by the particular disclosed embodiments described herein.

Methods of using the foregoing system(s) (including device(s), apparatus(es), assembly(ies), structure(s) or the like) are included; the methods of use can include using or assembling any one or more of the features disclosed herein to achieve functions and/or features of the system(s) as discussed in this disclosure. Methods of manufacturing the foregoing system(s) are included; the methods of manufacture can include providing, making, connecting, assembling, and/or installing any one or more of the features of the system(s) disclosed herein to achieve functions and/or features of the system(s) as discussed in this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Some embodiments have been described in connection with the accompanying drawings. Components can be added, removed, and/or rearranged. Orientation references such as, for example, “top” and “bottom” are for ease of ease of discussion and may be rearranged such that top features are proximate the bottom and bottom features are proximate the top. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In summary, various embodiments and examples of thermal thrombectomy devices, systems, and methods have been disclosed. Although the systems and methods have been disclosed in the context of those embodiments and examples, it will be understood by those skilled in the art that this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims that follow.

1. A thrombectomy device configured to remove a thrombus from a vein, the thrombectomy device comprising:
a plurality of shafts comprising an inner shaft, an inner expander shaft, and an outer expander shaft;
a collection device comprising a basket with a closed distal end coupled to the inner shaft and an open proximal end;
a cutting device comprising a frame, the frame comprising:
an open distal end coupled to the open proximal end of the collection device;
an open proximal end;
an interior lumen spanning between the open proximal end and the open distal end of the frame; and
one or more cutting elements disposed at the open proximal end;
an expansion mechanism comprising a distal portion, a proximal portion, and a plurality of members spanning between the proximal portion and the distal portion, the distal portion coupled to the inner expander shaft, the proximal portion coupled to the outer expander shaft, and the plurality of members coupled to the one or more cutting elements;
wherein the expansion mechanism is configured to expand the open proximal end of the cutting device with the proximal portion and distal portion moved toward each other such that the plurality of members move radially outward with the one or more cutting elements; and
wherein the expansion mechanism is configured to collapse the open proximal end of the cutting device with the proximal portion and the distal portion moved away from each other such that the plurality of members move radially inward with the one or more cutting elements.

2. The thrombectomy device of claim 1, wherein the inner expander shaft is configured to be distally advanced relative to the outer expander shaft to collapse the expansion mechanism.

3. The thrombectomy device of claim 1, wherein the inner expander shaft is configured to be proximally retracted relative to the outer expander shaft to expand the expansion mechanism.

4. The thrombectomy device of claim 1, wherein the one or more cutting elements comprise proximally-pointed cutting points.

5. The thrombectomy device of claim 1, wherein the frame of the cutting device comprises a plurality of struts coupled together to form diamond-shaped cells.

6. (canceled)

7. The thrombectomy device of claim 1, wherein the one or more cutting elements comprises four cutting elements.

8. (canceled)

9. The thrombectomy device of claim 1, further comprising a conductor configured to be heated, the conductor disposed proximate the one or more cutting elements to ease cutting the thrombus.

10. (canceled)

11. The thrombectomy device of claim 9, wherein the conductor is heated with electrical energy.

12. The thrombectomy device of claim 1, wherein the outer expander shaft includes a handle and the inner expander shaft includes a handle.

13. The thrombectomy device of claim 12, further comprising a threaded connection between the handle of the inner expander shaft and the handle of the outer expander shaft, wherein rotation of the handle of the inner expander shaft is configured to distally advance or proximally retract the inner expander shaft relative to the outer expander shaft.

14. A thrombectomy device comprising
a collection device with a mesh structure having a closed distal end and an open proximal end;
a cutting device comprising a frame, the frame comprising:
an open distal end coupled to the open proximal end of the collection device;
an open proximal end;
an interior passage spanning between the open proximal end and the open distal end of the frame; and
one or more cutting elements disposed at the open proximal end;
an expander coupled to the open proximal end, the expander configured to expand and collapse to expand and collapse the open proximal end of the cutting device, wherein the expander is configured to enable a clinician to adjust and maintain a cutting diameter of the one or more cutting elements.

15. The thrombectomy device of claim 14, wherein the expander is configured to enable the clinician to adjust the diameter of the cutting diameter along a continuum of diameters.

16. The thrombectomy device of claim 14, wherein the expander comprises a balloon that is configured to be inflated to expand the open proximal end of the cutting device and to be deflated to collapse the open proximal end of the cutting device.

17. The thrombectomy device of claim 14, further comprising a conductor configured to be disposed proximate the one or more cutting elements, the conductor configured to be heated to facilitate cutting a thrombus.

18. The thrombectomy device of claim 14, further comprising an inner expander shaft and an outer expander shaft, wherein the expander is coupled to the inner expander shaft and the outer expander shaft such that relative axial movement between the inner expander shaft and the outer expander shaft expand or collapse the expander.

19. The thrombectomy device of claim 18, wherein the expander comprises members configured to move radially outward with distal ends of the inner expander shaft and the outer expander shaft moved closer together.

20. The thrombectomy device of claim 14, wherein the one or more cutting elements comprises proximally directed cutting points.

21. The thrombectomy device of claim 20, wherein the cutting points provide the cutting diameter.

22. A method of removing a thrombus, the method comprising:
unsheathing a thrombectomy device distal of the thrombus;
proximally retracting an inner expander shaft relative to an outer expander shaft such that members of an expansion device coupled to the inner expander shaft and the outer expander shaft expand radially outward to expand a cutting diameter of a frame of a cutting device;
proximally retracting the thrombectomy device to bring the cutting diameter of the frame in contact with the thrombus to cut the thrombus;
receiving the cut thrombus through an interior lumen of the frame and into a collection basket; and
distally advancing the inner expander shaft relative to the outer expander shaft such that the members of the expansion device coupled to the inner expander shaft and the outer expander shaft collapse radially inward to collapse the cutting diameter of the frame.

23. The method of claim 22, further comprising heating a conductor disposed proximate the cutting diameter.

24-61. (canceled)
